(19) United States
(12) Patent Application Publication
(10) Pub. No.: US 2020/0281243 A1
(43) Pub. Date: Sep. 10, 2020

(54) DIETARY PRODUCT

(71) Applicants: Greg Hannon, London (GB); Simon Knott, London (GB); CANCER RESEARCH TECHNOLOGY LTD, London (GB)

(72) Inventors: Greg Hannon, London (GB); Simon James Knott, London (GB)

(73) Assignee: CANCER RESEARCH TECHNOLOGY LTD, London (GB)

(21) Appl. No.: 16/763,352

(22) PCT Filed: Nov. 13, 2018
(86) PCT No.: PCT/GB2018/053283

§ 371 Date: May 12, 2020

Related U.S. Application Data
(60) Provisional application No. 62/585,130, filed on Nov. 13, 2017.

(51) Int. Cl. A23L 33/175; A23L 33/00; A61K 31/198; A61P 35/04; A61K 38/00; A61K 38/50

(52) U.S. Cl. CPC A23L 33/175; A23L 33/30; A61K 38/50; A61P 35/04; A61K 38/005; A61K 31/198

(57) ABSTRACT

The present invention relates to dietary product comprising a plurality of amino acids, wherein the dietary product comprises all the essential amino acids and wherein the dietary product is substantially devoid of asparagine, processes of preparation of the product and methods and uses thereof in therapeutic applications such as the treatment of cancer.

### FIELD OF THE INVENTION

The present invention generally relates to the field of therapies for delaying or inhibiting metastasis and in increasing responsiveness to treatment in a subject with cancer. More particularly, the present invention relates to altering the levels of asparagine in the blood serum of a subject by reducing asparagine in the diet or by other means so as to delay or inhibit metastasis and/or prevent epithelial to mesenchymal transition. The invention also relates to the identification and treatment of patient populations with cancer at particular risk of the cancer metastasizing.

### BACKGROUND OF THE INVENTION

Cancer is a disease where cells undergo uncontrolled growth, growing and dividing beyond the normal limits of cell growth. These cells can invade and destroy surrounding tissues. Furthermore, cancer cells can metastasize, where they can spread to other areas of the body via the blood or lymphatic system.

Cancer treatment can involve surgery to remove the tumours, radiotherapy to reduce tumour size, or pharmacotherapy/chemotherapy, using drugs or other medicines to treat the cancers. Survival rates for cancers vary between cancer types; however, for cancer which has metastasized rates are especially low.

For example, the majority of women who succumb to breast cancer are not killed by the primary tumour, but by metastases that become apparent after the initial lesion has been removed. In order for cells to contribute to metastasis, they must leave the primary site, enter the vasculature, survive in the blood and then extravasate and colonize secondary sites (Vanharanta et al. 2013). Prior studies of a model of breast tumour heterogeneity, identified two clonal 4T1 sub-lines with the capacity to enter the vasculature through a non-invasive mechanism requiring vascular mimicry (4T1-E and -T) (Wagenblast et al. 2015 and Miller at al. 1983). However, these two clones differed greatly in their contribution to secondary lesions.

Thus, there is a need to identify drivers of metastasis and to provide medicaments which can delay or inhibit metastasis.

### BRIEF SUMMARY OF THE DISCLOSURE

The inventors have surprisingly discovered that asparagine synthetase (ASNS) expression in the primary tumour is strongly correlated with later metastatic relapse and that reducing asparagine blood serum levels in a subject with cancer can delay or inhibit metastasis. Furthermore, the inventors have surprisingly shown that reducing the availability of extracellular asparagine, through diet or otherwise, advantageously delays or inhibits metastasis. Dietary means of delaying or inhibiting metastasis may advantageously be complementary with existing cancer treatment regimens.

Accordingly, in a first aspect of the invention, the present invention provides a dietary product comprising a plurality of amino acids, wherein the dietary product comprises all the essential amino acids and wherein the dietary product is substantially devoid of asparagine. Suitably, the dietary product may comprise at least 12 amino acids.

Suitably, the dietary product may be substantially devoid of at least one or a plurality of additional non-essential amino acids selected from the group consisting of: glutamine, glycine, serine, cysteine, tyrosine and arginine.

Suitably, the dietary product may further comprise one or more macronutrients and/or one or more micronutrients.

Suitably, the dietary product may further comprise methionine, for example at a level of less than 25 mg/kg/day or less than 20 mg/kg/day or less than 18 mg/kg/day or less than 16 mg/kg/day.

Suitably, the product may be formulated to provide at least the recommended daily intake of essential amino acids based on average daily total protein consumption.

Suitably, the dietary product may be in the form of a solid or fluid. It may be formulated for oral administration as a meal replacement. Alternatively, it may be delivered intravenously.

In another, aspect, the present invention provides a process of preparing a dietary product of the invention, wherein the components are dissolved or dispersed in water and spray dried.

In a further aspect, the present invention provides a pharmaceutical composition comprising a dietary product of the invention or a dietary product produced in accordance with a process of the invention and a pharmaceutically acceptable carrier, excipient or diluent.

Suitably, the pharmaceutical composition may further comprise a therapeutic agent selected from: an inhibitor of cancer cell growth, an anti-metastatic agent, an immune checkpoint inhibitor, a radiotherapeutic agent and a chemotherapeutic agent. Suitably, the therapeutic agent may reduce asparagine levels in the blood.

Suitably, the therapeutic agent may be an asparagine synthetase inhibitor or L-asparaginase.

The present invention further provides a dietary product of the invention or a dietary product produced in accordance with the invention or a pharmaceutical composition of the invention for use in therapy.

In a further aspect, the present invention provides a medicament for use in delaying or inhibiting metastasis in a subject with cancer, wherein said medicament reduces asparagine levels in the blood (i.e. reduces asparagine bioavailability e.g. extracellular levels in the blood) of the subject with cancer.

Suitably, the medicament may be selected from the group consisting of:
a. a dietary product of the invention;
b. a dietary product produced in accordance with a process of the invention;
c. a pharmaceutical composition of the invention;
d. an asparagine synthetase inhibitor; or
e. L-asparaginase.

Suitably, the cancer may be selected from the group consisting of: breast cancer, colon cancer, squamous head and neck cancer, renal clear cell cancer and endometrial cancer

Suitably, the medicament may be a dietary product or pharmaceutical composition of the invention which may be formulated for co-administration or sequential administration with L-asparaginase.

Suitably, the medicament (e.g. the dietary product) may be used in combination with a therapeutic agent selected from: an inhibitor of cancer cell growth, an anti-metastatic agent, an immune checkpoint inhibitor, a radiotherapeutic agent and a chemotherapeutic agent.

Suitably, the subject may have been determined to have an expression level of asparagine synthetase which is higher than a control or a predetermined level. Suitably, the expression level of asparagine synthetase may be determined in a tumour sample.

Suitably, the subject may have been determined to have a level of serum asparagine which is higher than a control or a predetermined level.

Suitably, the subject may have a solid tumour.

In another aspect, the present invention relates to the use of a compound or composition in the manufacture of a medicament for delaying or inhibiting metastasis in a subject with cancer, wherein the compound or composition reduces asparagine levels in the blood of the subject (i.e. reduces asparagine bioavailability e.g. extracellular levels in the blood).

Suitably, the compound may be or composition may comprise:
a. a dietary product of the invention;
b. a dietary product produced in accordance with a process of the invention;
c. a pharmaceutical composition of the invention;
d. an asparagine synthetase inhibitor; or
e. L-asparaginase.

Suitably, the cancer may be selected from the group consisting of: breast cancer, colon cancer, squamous head and neck cancer, renal clear cell cancer and endometrial cancer.

Suitably, the dietary product or pharmaceutical composition may be formulated for co-administration or sequential administration with L-asparaginase.

Suitably, the compound or composition (e.g. dietary product) may be used in combination with a therapeutic agent selected from: an inhibitor of cancer cell growth, an anti-metastatic agent, an immune checkpoint inhibitor, a radiotherapeutic agent and a chemotherapeutic agent.

Suitably, the subject may have been determined to have an expression level of asparagine synthetase which is higher than a control or a predetermined level.

Suitably, the subject may have been determined to have a level of serum asparagine which is higher than a control or a predetermined level.

Suitably, the subject may have a solid tumour.

In a further aspect, the present invention provides a method of treating cancer in a subject, comprising administering a therapeutically effective amount of a medicament to said subject, wherein said medicament reduces asparagine levels in the blood of the subject with cancer (i.e. reduces asparagine bioavailability e.g. extracellular levels in the blood).

Suitably, the medicament may comprise:
a. a dietary product of the invention;
b. a dietary product produced in accordance with a process of the invention;
c. a pharmaceutical composition of the invention;
d. an asparagine synthetase inhibitor; or
e. L-asparaginase.

Suitably, the cancer may be selected from the group consisting of: breast cancer, colon cancer, squamous head and neck cancer, renal clear cell cancer and endometrial cancer.

Suitably, a therapeutically effective amount of the medicament (e.g. a dietary product or the pharmaceutical composition of the invention) may be co-administered or sequentially administrated with a therapeutically effective amount of L-asparaginase.

Suitably, a therapeutically effective amount of the medicament (e.g. dietary product) may be administered in combination with a therapeutic agent selected from: an inhibitor of cancer cell growth, an anti-metastatic agent, an immune checkpoint inhibitor, a radiotherapeutic agent and a chemotherapeutic agent.

Suitably, the subject may have been determined to have an expression level of asparagine synthetase which is higher than a control or a predetermined level.

Suitably, the subject may have been determined to have a level of serum asparagine which is higher than a control or a predetermined level.

Suitably, the subject may have a solid tumour.

Suitably, the dietary product may be the sole source of nutrition for the subject.

Suitably, the treatment may be administered over a period of at least 24 hours or until a therapeutic endpoint is observed.

Suitably, the medicament may be administered between 1 and 6 times a day.

Suitably, at least the recommended daily amount of essential amino acids may be met by the administration regimen of the dietary product each day.

In another aspect, the present invention provides the use of blood (e.g. serum) asparagine levels as a biomarker to identify a patient or patient population having a tumour which is at increased risk of metastasis.

In a further aspect, the present invention provides use of asparagine synthetase expression as a biomarker to identify a patient or patient population having a tumour which is at increased risk of metastasis. Suitably, the expression level may be determined in the primary tumour.

The present invention further provides a method of identifying a subject with cancer having an increased likelihood of metastasis comprising:
a) determining the level of asparagine in a biological sample (e.g. blood serum) isolated from the subject;
b) comparing the level of asparagine in the biological sample to a control sample or to a predetermined reference level of asparagine,
   wherein an increased level of asparagine in the biological sample compared to the control sample or compared to the predetermined reference level is indicative of increased likelihood of metastasis.

Suitably, the method may further comprise administering a therapeutically effective amount of a medicament to said subject, wherein said medicament reduces extracellular asparagine levels in the blood of the subject with cancer.

Suitably, the medicament may be or may comprise:
a. a dietary product of the invention;
b. a dietary product produced in accordance with a process of the invention;
c. a pharmaceutical composition of the invention;
d. an asparagine synthetase inhibitor; or
e. L-asparaginase.

The present invention further comprises a method of identifying a subject having an increased likelihood of responsiveness or sensitivity to a cancer treatment when fed a diet substantially devoid of asparagine or administered with L-asparaginase comprising:
a) determining the level of asparagine in a biological sample isolated from the subject;
b) comparing the level of asparagine in the biological sample to a control sample or to a predetermined reference level of asparagine,
   wherein an increased level of asparagine in the biological sample compared to the control sample or compared to the predetermined reference level is indicative of responsiveness or sensitivity to said cancer treatment when combined with a diet substantially devoid of asparagine or administered with L-asparaginase.

Suitably, the method may further comprise administering a therapeutically effective amount of the dietary product of the invention or the dietary product produced in accordance with the invention or the pharmaceutical composition in accordance with the invention or L-asparaginase in combination with a chemotherapeutic agent when the subject is identified as having an increased likelihood of responsiveness or sensitivity to a cancer treatment when fed a diet substantially devoid of asparagine or administered with L-asparaginase.

Suitably, the biological sample may be a blood sample (e.g. serum).

The present invention further provides a method of determining the likelihood of metastatic relapse in a subject having a cancer comprising:
a) determining the level of asparagine synthetase in a biological sample isolated from the subject;
b) comparing the level of asparagine synthetase in the biological sample to a control sample or to a predetermined reference level of asparagine,
   wherein an increased level of asparagine synthetase in the biological sample compared to the control sample or compared to the predetermined reference level is indicative of an increased likelihood of metastatic relapse.

In a further aspect, the present invention provides a method of reversing epithelial to mesenchymal transition in a subject or preventing epithelial to mesenchymal transition in a subject with cancer comprising administering a therapeutically effective amount of the dietary product of the invention or the dietary product produced in accordance with a process of the invention or a pharmaceutical composition of the invention or L-asparaginase to a subject in need thereof.

These and other aspects are expanded on the in the detailed description.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### BRIEF DESCRIPTION OF THE DRAWINGS

Embodiments of the invention are further described hereinafter with reference to the accompanying drawings, in which:

FIG. 1 shows identification of metastatic drivers. a) Relative proportions of 4T1-E and -T cells extracted from the lungs of NSG mice, into which mixtures of cells were introduced via tail vein at different concentrations. Each bar represents a sample or independent mouse or sample. b) Expression level of genes identified as over-expressed in 4T1-T as compared to 4T1-E in the primary tumours of patients with different disease subtypes (edges of the box are the 25^{th} and 75^{th} percentiles and error bars extend to the values q3+w(q3−q1) and q1−w(q3−q1), where w is 1.5 and q1 and q3 are the 25^{th} and 75th percentiles and this is also true for c, ANOVA p-value <0.0001) c) Expression level of the same genes in disease free survivors and patients with relapse to the lung rank-sum p-value <0.01) d) RNAi screening scheme to identify drivers of invasion in vitro and extravasation and colonization in vivo (n=5 mice or n=2 matrigel 6-well invasion chambers per shRNA pool, gene-level hits calls with empirical bayes moderated t-test FDR<0.05). e) Overlap between genes identified in each arm of the RNAi screen depicted in d (hypergeometric test p-value <0.0001).

FIG. 2 shows validation of Asparagine Synthetase as a driver of invasion and metastasis. a) Quantification of metastases in the lungs of mice that were intravenously injected with Asns-silenced or -expressing 4T1-T cells (n=10 mice per cell line, edges of the box are the 25^{th} and 75^{th} percentiles and error bars extend to the values q3+w(q3−q1) and q1−w(q3−q1), where w is 1.5 and q1 and q3 are the 25^{th} and 75^{th} percentiles and this is also true for e, f and g, rank-sum test p-value <0.001). b) Representative images of the lungs described in a c) Representative images of collection wells of the matrigel assay after Asns silenced and -expressing cells were applied 24 hours prior (n=3 invasion chambers per cell line) d) Tumour volumes resulting from the orthotopic injection of the cells described in a (n=10 mice per cell line, error bars are the 25^{th} and 75^{th} percentiles) e) Relative abundance of CTCs in animals corresponding to the tumours described in d (n=4 mice per cell line, rank-sum p-value <0.05). CTC abundance was measured by qPCR for mCherry, which is expressed from the retroviral shRNA delivery vectors, from whole blood genomic DNA f) Quantification of metastases in H&E stained lung sections, from mice described in e (rank-sum p-value <0.0002) g) Diameters of the metastases described in f.

FIG. 3 shows extracellular asparagine availability drives invasion and metastasis. a) HPLC based quantification of cellular free asparagine levels for Asns-silenced and -expressing cells (n=3 replicates per cell line) b) Quantification of parental 4T1 cell invasion rates, as measured by the matrigel invasion assay, in culture media supplemented with the indicated non-essential amino acids (n=5 invasion chambers, rank-sum p-value <0.01) c) Quantification of lung metastases in animals that had been injected with Asns-silenced or -expressing 4T1-T cells. For each line half of the animals were administered PBS while the other half were administered L-asparaginase (n=10 mice per condition, error bars are the 25th and 75th percentiles and this is also true for e and f. rank-sum p-value <0.0005 for L-asparaginase vs. control for each line and for Asns-silenced vs. -unsilenced cells in each drug condition) d) Representative H&E stained lung sections from each experimental condition represented in c. e) Quantification of lung metastases in animals that had been injected with Asns-silenced or -expressing 4T1-T cells. Animals were administered a diet with either 0%, 0.6% or 4% asparagine content for the duration of the experiment (n=10 mice per condition, rank-sum p-value <0.0005 for Asns-silenced vs. -expressing cells across all diets, shRenilla cells with 0% vs. 0.6% vs. 4% diets, and for shAsns-1 and -2 with 4% vs. 0% diets, rank-sum p-value <0.05 for shAsns-1 and shAsns-2 with 0.6% vs. 0% or 0.6% vs 4% diets) f) Mass-spectrometric quantification of the asparagine levels in the mammary gland, serum and lungs of animals that were administered L-asparaginase or PBS (relative abundance normalized by total metabolite peak area, n>8 tissue sections per condition, rank-sum p-value <0.005 for PBS vs. L-asparaginase across all tissues, rank-sum p-value <0.05 for Mammary gland vs. Lung and rank-sum p-value <0.0005 for Serum vs. Lung and Serum vs. Mammary gland).

FIG. 4 shows asparagine availability regulates epithelial-to-mesenchymal transition a) Amino acid enrichment analysis of protein vs. RNA level expression changes induced by Asns-silencing. Amino acids with negative correlations are enriched in proteins where protein level changes are less than would be expected by corresponding RNA level changes. Positive correlations indicate the amino acid is enriched in genes where protein level changes exceed those predicted by changes in RNA quantity. b) Relative protein abundances of EMT-up proteins in Asns-silenced and -expressing 4T1-T cells (n=3 replicates per cell line, edges of the box are the 25^{th} and 75^{th} percentiles and error bars extend to the values q3+w(q3−q1) and q1−w(q3−q1), where w is 1.5 and q1 and q3 are the 25^{th} and 75^{th} percentiles and this is also true for c, d and h, rank-sum p-value <0.05) c) Relative protein abundances of EMT-up proteins in parental 4T1 cells when grown in normal and Asparagine-supplemented media (n=3 replicates per cell line, rank-sum p-value <0.05). d) Relative expression levels of EMT-up and EMT-down genes in lung metastases vs. primary tumours derived from orthotopic injection of parental 4T1 cells (n=4 mice, sign-rank p-value <0.001 for EMT-up genes) e) Representative images of IHC stainings for Twist1 and Cdh1 in orthotopic tumours that were derived from Asns-silenced and -expressing 4T1-T cells. f) Quantification of all Twist1 stainings, described in e (n=5 tumour sections and n>5 lung metastases, error bars represent 1 standard deviation and this is also true for g, rank-sum p-value <0.01 and <0.05 for Asns-silenced vs. -expressing tumors and metastases, respectively). g) Quantification of all Cdh1 staining, described in e (n=5 tumour sections and n=9 lung metastases, rank-sum p-value <0.01 and <0.05 for Asns-silenced vs. -expressing tumors and metastases, respectively). h) Relative expression levels of Twist1, Cdh1, EMT-up and EMT-down genes in cells that were isolated from tumours and lungs that were derived from Asns-silenced vs. Asns-expressing 4T1-T cells (n=2 replicates per condition, sign-rank p-value <0.05 for EMT-down genes in the tumour and sign-rank p-value <0.001 for EMT-up genes in the lung, Cdh1 and Twist1 were differentially expressed in both tissues, DESeq FDR<0.05).

FIG. 5 shows primary analysis of ASNS expression levels in patient data. For each gene that was identified as up-regulated in 4T1-T vs. -E, a prognostic value was calculated using three different datasets. One consisted of gene expression measurements in three patient-matched Basal tumour and metastasis pairs (Patients A1, A7 and A11). Here genes were classified as correlated with progression if expression was higher in each of the metastases and negatively correlated if expression was higher in each of the primaries. The other two datasets consisted of primary tumour gene expression profiles with matched outcomes. For the UNC254 patient dataset, the site of relapse was not available and genes were deemed positively correlated with progression if they had significant relapse free survival hazard ratios >1, and negatively correlated if these ratios were significant (cox p-value <0.05) and <1. As the UNC855 dataset also had site of relapse information, here both relapse free and lung relapse free survival (RFS and LRFS) hazard ratios were used to classify genes as positively or negatively correlated with progression based on the same criteria that were used for the UNC254 data. Shown are genes with human orthologues that were measured in the different datasets.

FIG. 6 shows secondary analysis of ASNS expression levels in patient data. a) Expression level of Asparagine Synthetase in the primary tumours of patients with different disease subtypes (edges of the box are the 25^{th} and 75^{th} percentiles and error bars extend to the values q3+w(q3−q1) and q1−w(q3−q1), where w is 1.5 and q1 and q3 are the 25^{th} and 75^{th} percentiles and this is also true for b, ANOVA p-value <0.0001). b) Expression level of Asparagine Synthetase in the primary tumours of patients with non-specific relapse and relapse to the lymph node, bone, brain, liver or lung in comparison to expression levels in patients without relapse to each corresponding site (rank-sum p-value <0.005). c) Analysis of ASNS in 3 additional breast cancer patient sets (MDACC, METRABIC and TCGA). Shown are survival plots and relevant statistics (cox p-value <0.001). d) Analysis of ASNS in the TCGA Pan Cancer expression data. Shown are survival plots and relevant statistics for the 10 non-breast solid tumours represented in the dataset (cox p-value <0.05 for Colon, Squamous Head and Neck, Renal Clear Cell and Endometrial cancers). e) Analysis of ASNS across all tumours represented in the TCGA Pan Cancer dataset (cox p-value <0.001).

FIG. 7 shows primary validation of Asns as a driver of invasion and metastasis. a) Quantification of matrigel invasion capacity for Asns-silenced and -expressing 4T1-T cells (n=3 replicates per cell line). b) Quantification of mCherry-positive 4T1-T cells after roughly 50% of cells were infected with mCherry-expressing constructs harboring shRNAs targeting Renilla Luciferase and Asparagine Synthetase. Cells were grown during the 24-hour period that the matrigel invasion assay described in FIG. 2c was being performed (n=3 replicates per cell line). c) Violet cell-labelling intensity of Asns-silenced and -expressing 4T1-T cells, relative to the initial population. Cells were grown during the 24-hour period that the matrigel invasion assay described in FIG. 2c was being performed (n=3 replicates per cell line). d) Representative H&E stained sections of the tumours described in FIG. 2d, where Asns-silenced and -expressing 4T1-T cells were orthotopically injected into NSG mice. e) Representative H&E stained sections of the lungs from mice harboring the tumours shown in d.

FIG. 8 shows secondary validation of Asns as a driver of invasion and metastasis. a) Volume measurements of tumours resulting from orthotopic injection of Asns-silenced and -expressing parental 4T1 cells (n=10 mice per cell line, edges of the box are the 25th and 75th percentiles and error bars extend to the values q3+w(q3−q1) and q1−w(q3−q1), where w is 1.5 and q1 and q3 are the 25^{th} and 75^{th} percentiles and this is also the case for b. b) Quantification of lung metastases corresponding to the tumours described in a (rank-sum p-value <0.002). c) Volume measurements of tumours resulting from orthotopic injection of parental 4T1 cells with basal (Empty) or enforced (Asns) expression of Asns (n=10 mice per cell line, edges of the box are the 25th and 75th percentiles and error bars extend to the values q3+w(q3−q1) and q1−w(q3−q1), where w is 1.5 and q1 and q3 are the 25th and 75th percentiles and this is also the case for d, f and g). d) Quantification of lung metastases corresponding to the tumours described in c (rank-sum p-value <0.02). e) Representative H&E stained sections of the lungs described in d. f) Volume measurements for tumours resulting from orthotopic injection of MDA-MB-231 cells with basal (Empty) or enforced expression of ASNS (n=10 mice per replicate). g) Quantification of lung metastases corresponding to the tumours described in f (rank-sum p-value <0.02). h) Quantification of matrigel invasion for the MDA-MB-231 derived cell lines described in f (n=3 invasion chambers per cell line). i) Representative images of the collection wells for the invasion assays described in h.

FIG. 9 shows primary validation that extracellular asparagine availability impacts invasion and metastasis. a) Percentage makeup of cellular free amino acids were measured with HPLC for Asns-silenced and -expressing cells. Shown are the log 2-fold-changes in these percentages per amino acid (n=3 replicates per cell line, empirical-bayes moderated t-test FDR<0.05). b) Quantification of mCherry-positive 4T1-T cells after roughly 50% of cells were infected with mCherry-expressing constructs harboring shRNAs targeting Renilla Luciferase and Asns. After infection cells were grown in L-asparagine or D-asparagine supplemented media and mCherry percentages were measured at 48 and 96 hours (n=3 replicates per cell line, error bars represent 1 standard deviation and this is also true for c. c) Quantification of matrigel invasion for Asns-silenced and -expressing cells when assayed in media that has been supplemented with and without L-asparagine. d) HPLC quantification of cellular free amino acid percentages for parental 4T1 cells when media is supplemented with each of the non-essential amino acids that is lacking in the DMEM culture media (n=3 replicates per cell line) e) Quantification of MDA-MB-231 matrigel invasion rates under the same conditions as was described in FIG. 3b (n=5 invasion chambers per condition, rank-sum p-value <0.001) f) HPLC quantification of cellular free amino acid percentages for MDA-MB-231 cells when cultured in the media conditions described in d (n=3 replicates per cell line). g) Violet cell-labeling intensity of parental 4T1 cells when they have been grown in asparagine-lacking and -supplemented media for the 24 hour period that the matrigel invasion assay described in FIG. 3b was being performed (n=3 replicates per cell line). h) Violet cell-labeling intensity of MDAMB-231 cells when they have been grown in asparagine-lacking and -supplemented media for the 24-hour period that the matrigel invasion assay described in e was being performed (n=3 replicates per cell line).

FIG. 10 shows secondary validation that extracellular asparagine availability impacts invasion and metastasis. a) Tumour volumes resulting from the orthotopic injection of parental 4T1 cells. Half of the mice received L-asparaginase while the other half received an equivalent volume of PBS at the same injection rate (n=10 mice per condition, edges of the box are the 25^{th} and 75^{th} percentiles and error bars extend to the values q3+w(q3−q1) and q1−w(q3−q1), where w is 1.5 and q1 and q3 are the 25^{th} and 75^{th} percentiles and this is also the case for b, d e and f. b) Quantification of lung metastases detected in the animals described in a) (rank-sum p-value <0.0002). c) Representative H&E stained lung sections as described in a. d) Tumour volumes corresponding to the lung metastases described in FIG. 3c & FIG. 3d, where Asns-silenced and -expressing 4T1-T cells were injected into mice. Half of the mice received L-asparaginase while the other half received an equivalent volume of PBS at the same injection rate (n=10 mice per condition, rank-sum p-value <0.05 for Asns-silenced vs. -expressing cells in L-asparaginase treated mice). e) Tumour volumes resulting from the orthotopic injection of ASNS-silenced and -expressing MDA-MB-231 cells and subsequent treatment of the injected animals with L-asparaginase or PBS (n=10 mice per cell line). f) Lung metastases corresponding to the orthotopic tumours described in e (rank-sum p-value <0.05 for Asns-silenced vs. -expressing cells under both treatments and for PBS vs. L-asparaginase treated animals for each cell line).

FIG. 11 shows tertiary validation that extracellular asparagine availability impacts invasion and metastasis. a) Asparagine content (%) in the serum free amino acid pool. for mice that have been fed 0%, 0.6% or 4% asparagine diets (n=5 mice per diet, edges of the box are the 25th and 75th percentiles and error bars extend to the values q3+w(q3−q1) and q1−w(q3−q1), where w is 1.5 and q1 and q3 are the 25th and 75th percentiles and this is also the case for b, d, e and h, rank-sum p-value <0.05 between each diet). b) Volumes of orthotopic tumours corresponding to the lung metastases described in FIG. 3e, where Asns-silenced and -expressing 4T1-T cells were orthotopically injected into mice fed 0%, 0.6% and 4% asparagine diets (n=10 mice per condition). c) Representative images of the lung metastases described for FIG. 3e, which also correspond to the mice described in b. d) Volumes of tumours resulting from the orthotopic injection of parental 4T1 cells into mice fed 0%, 0.6% or 4% asparagine diets (n=10 mice per diet). e) Quantification of metastases in the lungs of the animals described in d (rank-sum p-value <0.05 between each diet) f) Representative images of H&E stained sections of the lungs described in e. g) Relative expression of Asns in the mammary gland, serum and lungs of mice that have been treated with L-asparaginase or PBS, as measured by qPCR with two primer pairs (n=4 per condition, error bars extend to 1 standard deviation, rank-sum p-value <0.05 between tissues). h) Reads per Kilobase of transcript per Million mapped reads (RPKM) expression measurements for ASNS in human breast, lung and whole blood samples (n>90 for each tissue, rank-sum p-value <0.05 between tissues).

FIG. 12 shows primary validation that asparagine availability regulates EMT. a) Protein-level changes between Asns-silenced and expressing cells when genes are stratified by transcription-level changes (top and bottom 10% of genes based on level of change in Asns-silenced cells, Gene-up and Gene-down, respectively) and asparagine content (top and bottom 10% of genes based on asparagine content, Asp-high and -low, respectively, edges of the box are the 25^{th} and 75^{th} percentiles and error bars extend to the values q3+w(q3−q1) and q1−w(q3−q1), where w is 1.5 and q1 and q3 are the 25^{th} and 75^{th} percentiles, rank-sum p-value <0.001 for both individual variables, and rank-sum p-value <0.05 for interacting variables). b) Amino acid enrichment analysis of genes based on RNA and protein level expression changes induced by Asns-silencing. Amino acids with negative correlations are those whose abundance correlates negatively with corresponding RNA or protein level changes induced by silencing, while those with positive correlations correlate positively with these expression changes. c) Amino acid enrichment in murine EMT-up proteins. d) Amino acid enrichment in human EMT-up proteins. e) Asparagine enrichment analysis of epithelial-to-mesenchymal promoting protein orthologs in the 126 species listed in the Orthologous MAtrix database that harbor at least 10 orthologs (sign-rank p-value <1.0×10−13 for all species and rank-sum p-value <9.0×10−9 for mammals vs. other species).

FIG. 13 shows secondary validation that asparagine availability regulates EMT. a) Transcription-level changes in EMT-up and -down genes that occur in response to Asns-silencing in 4T1-T cells (n=2 replicates per condition, edges of the box are the 25th and 75th percentiles and error bars extend to the values q3+w(q3−q1) and q1−w(q3−q1), where w is 1.5 and q1 and q3 are the 25^{th} and 75^{th} percentiles and this is also the case for b, c, f and g, sign-rank p-value <0.001 for EMT-up genes). b) Transcription-level changes in EMT-up and -down genes that occur in response to the media of parental 4T1 cells being supplemented with asparagine (n=2 replicates per condition, sign-rank p-value <0.005 for EMT-up genes). c) Volumes of tumours resulting from orthotopic injection of Tgf-ß-silenced and -expressing 4T1-T cells (n=10 mice per cell line) d) Percent Twist1 positive regions based on IHC staining of sections from tumours described in c (n=5 tumour sections per cell line, error bars extend to 1 standard deviation and this is also true for e, rank-sum p-value <0.01 for Asns-silenced vs. -expressing cells). e) Percent Cdh1 positive regions based on IHC staining of sections from tumours described in c (n=5 tumour sections per cell line, rank-sum p-value <0.01 for Asns-silenced vs. -expressing cells). f) Quantification of metastases resulting from the tumours described in c (rank-sum p-value <0.05). g) Quantification of metastases resulting from intravenous injection of Tgf-ß-silenced and -expressing cells (10 mice per cell line, rank-sum p-value <0.05).

FIG. 14 shows tertiary validation that asparagine availability regulates EMT. a) Representative images of IHC staining for Twist1 and Cdh1 on sections from lungs described in FIG. 4f & g. where mice were injected orthotopically with Asns silenced and -expressing 4T1-T cells. b) Relative Twist1 expression, as measured by qPCR, in the tumours and lungs described in a (n=2 tumours and lungs per cell line, error bars extend 1 standard deviation, and this is also true for c, d, e, f and g. c) Relative Cdh1 expression, as measured by qPCR, in the tumours and lungs described in a (n=2 tumours and lungs per cell line). d) Quantification of Twist1-positive regions in the tumours resulting from orthotopic injection of Asns-expressing and -silenced cells into animals that are treated with PBS or L-asparaginase (n=5 tumour sections per condition, rank-sum p-value <0.01). e) Quantification of Cdh1-positive regions in the tumours resulting from orthotopic injection of Asns-expressing and -silenced cells into animals fed a 0%, 0.6% or 4% asparagine described in d (n=5 tumour sections per condition, rank sum p-value <0.01). f) Quantification of Twist1-positive regions in tumours resulting from orthotopic injection of Asns-expressing and -silenced cells into mice fed a 0%, 0.6% or 4% asparagine diet (n=5 tumour sections per condition, rank-sum p-value <0.01 between Asns-silenced and -expressing cells and between diets). g) Quantification of Cdh1-positive regions in the tumours described in f (n=5 tumour sections per condition, rank-sum p-value <0.01 between Asns-silenced and -expressing cells and between diets). h) Images of cultured cells after they were isolated from the tumours and metastases described in a.

### DETAILED DESCRIPTION

The inventors have surprisingly found that a diet substantially devoid of asparagine can have utility in delaying or inhibiting metastasis in a subject with a proliferative disorder such as cancer. Specifically, removing asparagine from the diet of mice strongly reduced metastasis from orthotopic tumours. Without wishing to be bound by theory, the inventors have shown that asparagine limitation reduces the production of proteins that promote the epithelial to mesenchymal transition, this may be one potential mechanism by which the availability of asparagine regulates metastatic progression. Furthermore, asparagine limitation may reduce cancer stem call phenotype in a subject.

By “bioavailability” it is meant the proportion of asparagine which enters the circulation.

Suitably, the present invention may involve partly or completely substituting the normal diet of a subject suffering from cancer with a prescribed diet substantially devoid of asparagine. Such a diet may potentially be achieved by the provision of a dietary product as detailed herein, or by two or more dietary supplements which can be administered simultaneously or sequentially. Potentially, such a diet may be further supplemented through proper food selection, using ingredients currently available such that the diet remains substantially devoid of asparagine.

Suitably, the dietary product may be formulated such that it provides the required daily intake of essential amino acids. A person of ordinary skill in the art would readily recognise that this can be achieved by in various ways and will depend on the administration regimen for the dietary product. For example, if the dietary product were to be administered in a single dose per day, then the dose would comprise at least the daily recommended amounts of essential amino acids, whereas if administered 3 times a day then the three administrations combined would provide at least the daily recommended amounts of the essential amino acids.

Suitably, the diet or other medicaments which reduce serum asparagine levels may be used prior to cancer treatment to sensitise the cancer cells to further therapy. The present inventors have surprisingly shown that dietary asparagine content correlates positively with the epithelial to mesenchymal signature in the primary tumour. By reducing asparagine levels in cancer cells, epithelial to mesenchymal transition (EMT) can be prevented or reversed, increasing the sensitivity of cancer cells to therapy.

### Dietary Product

In a first aspect of the present invention, there is provided a dietary product comprising a plurality of amino acids, wherein the dietary product comprises all the essential amino acids and wherein the dietary product is substantially devoid of at least asparagine.

By “essential amino acids” it is meant methionine, leucine, phenylalanine, isoleucine, valine, lysine, threonine, histidine and tryptophan.

“Dietary product” refers to a composition comprising one or more essential amino acids or salts or esters thereof, that is used in a food product, or used or consumed in combination with a food product, to provide a desired level of the amino acid(s) or salt or esters thereof to the subject consuming the dietary product. The dietary ingredients in these products may include: vitamins, minerals, herbs or other botanicals, amino acids, and substances such as enzymes, organ tissues, glandulars, and metabolites. In the present invention, the dietary product may be formulated to comprise all the essential amino acids in a single composition or amongst a combination of dietary supplements which combine to provide all the essential amino acids over e.g. the course of a day. Suitably, the dietary supplements are substantially devoid of asparagine.

In some embodiments, the dietary product is the sole source of exogenous amino acids consumed by the subject as part of their diet. Suitably, in some aspects, the dietary product may be intended to substantially or solely replace a subject's diet. Hence, in some aspects, the dietary product may be a complete meal replacement for the subject.

Advantageously, replacement of consumption of usual sources of amino acids such as protein with a dietary product of the invention will yield a diet substantially devoid of asparagine. This may provide therapeutical benefits to a cancer subject.

As used herein, in accordance with all aspects of the invention, the term “subject” preferably refers to a mammalian animal, including a human, a veterinary or farm animal, a domestic animal or pet, and animals normally used for clinical research, including non-human primates, dogs and mice. More specifically, the subject of the present invention may be a human.

Suitably, the subject may have a population of cells with a cancer stem cell phenotype. Cancer stem cells are known in the art and may be involved not only in tumor recurrence but also is tumorigenicity, metastization and drug resistance.

Suitably, the dietary product may comprise at least 9 amino acids. Suitably, the dietary product may comprise at least 10 or at least 11 or at least 12 or at least 13 or at least 14 or at least 15 or at least 16 or at least 17 or 18 amino acids. Suitably, the dietary product may comprise 9 to 18 amino acids or 12-18 amino acids, or 12-17 amino acids or 13-17 amino acids or 14-17 amino acids, for example.

Suitably, the dietary product may be substantially devoid of one or more additional non-essential amino acids. Additional amino acids which may be substantially devoid in the dietary product may comprise (or consist essentially thereof or consist of) two or more of the following amino acids: glycine, serine, cysteine, tyrosine, proline and arginine. Alternatively, the dietary product may be devoid of at least two or at least three or at least four or at least five or at least six of the following amino acids in addition to asparagine: glycine, serine, cysteine, tyrosine, proline, arginine, alanine, aspartic acid, glutamic acid, and glutamine.

In this context, by “consist essentially thereof” it is meant that that the dietary product may not lack further amino acids which have a material effect on the dietary product on the invention. By “material effect” it is meant a significant therapeutic effect which may be measured as one of the following: a) a significant effect on the specificity for cancer as opposed to healthy cells; b) a significant effect on the delay or inhibition of metastasis; c) a significant effect on the toxicity of cancer cells or d) any combination of a)-c). In some aspects, this may be measured by comparing the dietary product with and without a particular amino acid and determining whether the lack of the amino acid has a material effect.

WO 2017/144877 discloses that a diet or a dietary product substantially devoid in serine and/or glycine is useful to reduce proliferation and/or cancer cell survival. This patent application also shows that: a dietary product substantially devoid of glycine, serine and cysteine is effective in inhibiting cancer cell proliferation and increasing cancer cell death in numerous cancer cell lines including colorectal (such as in HCT116 and RKO), liver (HepG2), osteosarcoma (U2OS) and breast (MDA MB 231) cancer; a dietary product substantially devoid of glycine, serine and arginine is surprisingly effective in inhibiting cancer cell proliferation and/or increasing cancer cell death in colorectal cells lines (such as RKO and HCT116); a dietary product substantially devoid of glycine, serine and tyrosine is surprisingly effective in inhibiting cancer cell proliferation and/or increasing cancer cell death in colorectal cells lines (such as RKO and HCT116) and that further beneficial effects occur when the diet is substantially devoid of cysteine. Thus, it would be beneficial for the dietary product of the invention to be substantially devoid of any one or more of these non-essential amino acids in addition to be being substantially devoid of asparagine.

The dietary product may further comprise methionine at a level of less than 25 mg/kg body weight of the subject/day or less than 20 mg/kg/day or less than 18 mg/kg/day or less than 16 mg/kg/day.

A dietary product of the invention may be formulated to provide at least the recommended daily intake of essential amino acids based on average daily total protein consumption, unless otherwise stated herein.

The recommended daily intake of essential amino acids by the Institute of Medicine, as based on average daily total protein consumption, is: Histidine 18 mg/g protein consumed; isoleucine 25 mg/g protein consumed; leucine 55 mg/g protein consumed, lysine 51 mg/g protein consumed, methionine and cysteine combined 25 mg/g protein consumed; phenylalanine and tyrosine combined 47 mg/g protein consumed, threonine 27 mg/g protein consumed, tryptophan 7 mg/g protein consumed and valine 32 mg/g protein consumed. Tyrosine and cysteine are non-essential amino acids. Where a dietary product of the invention is substantially devoid of either tyrosine and/or cysteine, the dietary product is adjusted such that the dietary product is formulated to provide methionine in an amount of at least 25 mg/g protein consumed and phenylalanine in an amount of at least 47 mg/g protein based on average daily protein consumption.

Suitably, a dietary product “restricted” in cysteine is one which provides less that is formulated to provide less than the recommended daily intake of cysteine based on average daily protein consumption. For example, a dietary product restricted in cysteine may be one which provides less than 20 mg/g protein consumed or less than 15 mg/g protein consumed or less than 10 mg/g protein consumed or less than 5 mg/g protein consumed.

Suitably, the dietary product may be formulated to provide a restricted level of total non-essential amino acids per gram of protein consumption. For example, the combined daily intake of non-essential amino acids may be equivalent to the diet being substantially devoid of at least one or at least two or at least three or at least four of at least five or at least six or at least seven non-essential amino acids compared with the recommended daily intake of total non-essential amino acids per gram of protein consumed.

The institute of medicine recommends that protein is consumed at a rate of 0.8 grams per kilogram per day of body weight for adults for example. The dietary product may be formulated to provide at least 0.8 grams protein per kg body weight during recommended daily consumption of the product.

Suitably, the dietary product of the invention may be formulated to provide these above recommended levels. For example, one or more amino acids may be formulated in the dietary product to provide at least 2, 3, 4, 5, or 6 times the daily average intake based on average daily total protein consumption.

Suitably, the amino acids present in the dietary product of the invention may be amino acids in free form, in prodrug form, salts or amino acid esters. Amino acids with one or more N-terminal or C-terminal modification, and homopolymer, homodimer, heteropolymer and heterodimer forms may also be contemplated.

Suitably, the dietary product may be formulated to be administered from once to eight times daily. Preferably, once to four times daily. Thus, the dietary product may be formulated to an appropriate unit dosage form.

The dietary product of the invention may further comprise one or more macronutrients and/or micronutrients.

Guidance on macronutrients and suggested recommended daily amounts may be found in the Dietary Reference Intakes for Energy, Carbohydrate, Fiber, Fat, Fatty Acids, cholesterol, protein and amino acids released by the Institute of Medicine September 2002.

A non-exhaustive list of macronutrients which may be additional components of the dietary product include: carbohydrate, fiber and fat (such as n-6 polyunsaturated fatty acids, n-3 polyunsaturated fatty acids, saturated and trans fatty acids and cholesterol).

A non-exhaustive list of micronutrients includes Vitamin A, Vitamin C, Vitamin D, Vitamin E, Vitamin K, Thiamin, Riboflavin, Niacin, Vitamin B6, folate, Vitamin B12, Pantothenic acid, biotin, choline, calcium, chromium, copper, fluoride, iodine, iron, magnesium, molybdenum, phosphorus, selenium, zinc, potassium, sodium, and chloride. Suitably the dietary product may be formulated to provide these in acceptable or recommended daily intake amounts as detailed in the publication “Dietary Reference Intakes: RDA and AI for Vitamins and Elements”, NAS. IOM. Food and Nutrition Board.

The dietary product described herein contain an imbalance of amino acids generally in the form of a deficiency of two or more non-essential amino acids, optionally complemented by a surplus of one or more other amino acids. For example, a substantially devoid amino acid may be at least 10, 15, 20, 30, 45, 50, 100, or 1000 times lower than the average abundance of the other amino acids. Foods that are low in protein but rich in other nutrients, such as fruits, vegetables and certain nuts can be consumed following a dietician's recommendation, making sure the dietary amino acid intake ratios are kept at the intended ratios. This diet (comprising dietary products of the invention and, optionally other sources of nutrition substantially devoid of asparagine) is intended to be consumed alone or in combination with drug therapies, such as those that have anti-cancer activity.

In some embodiments, the dietary product of the invention is formulated across two or more dietary supplements which together provide a dietary product of the invention. These may be administered simultaneously or sequentially to said subject, such that the combined average diet provided by the dietary supplements provides a dietary product of the invention. This may be advantageous to add variety to the subject's diet.

Dietary products may be provided in the form of a powder, a gel, a solution, a suspension, a paste, a solid, a liquid, a liquid concentrate, a powder which may be reconstituted, a shake, a concentrate, a pill, a bar, a tablet, a capsule or a ready-to-use product. It is contemplated that a dietary product can also be a pharmaceutical composition when the supplement is in the form of a tablet, pill, capsule, liquid, aerosol, injectable solution, or other pharmaceutically acceptable formulation. Suitably, the dietary product may be a beverage. Suitably, the beverage may be administered 2 to 6 times a day.

Suitably, the dietary product may not be a naturally occurring food.

Suitably, the dietary product may comprise additional compounds to the specified amino acids. Suitably, such additional compounds may not aid de novo synthesis of the substantially devoid amino acids.

As used herein “substantially devoid” in reference to an amino acid means completely or very nearly free (such as trace amounts) of that amino acid.

Optionally, administration may be by an intravenous route. Optionally, parenteral administration may be provided in a bolus or by infusion.

Suitably, the dietary product may be:
a) A tube fed enteral nutritional product (such as a naso-gastric nutritional product, which may be administered via a NG tube; a naso-jejunal nutritional product, which may be administered via a NJ tube; or a PEG (percutaneous endoscopic gastrostomy) tube nutritional product);
b) a parenteral nutrition product (which may be administered by central venous administration, e.g. via dedicated lumen on a venous catheter); or
c) an IV infusion product.

Preferably the administration may be as a food or beverage.

In certain embodiments, the diet or dietary product of the invention is administered over a time period of at least 1 day, 2 days, 3 days, 4 days, 5 days, 6 days, 7 days, at least 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, or until a therapeutic endpoint is observed, e.g., a statistically significant reduction is asparagine levels in the blood serum or a reduction in the epithelial to mesenchymal (EMT) phenotype of cancer cells is observed.

The present invention further provides a process of preparing a dietary product of the invention, wherein the amino acids are dissolved or dispersed in water and spray dried.

Suitably, the amino acids may be mixed with additional components such as macronutrients and micronutrients. Binders, emulsifiers or other ingredients suitable for human or animal consumption may be added as desired.

### Pharmaceutical Composition

In another aspect, the present invention provides a pharmaceutical composition comprising a dietary product of the invention or a dietary product produced in accordance with the invention and a pharmaceutically acceptable carrier, excipient or diluent.

Conventional procedures for the selection and preparation of suitable pharmaceutical formulations are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitably, the pharmaceutical composition is formulated to provide a therapeutically effective amount of the dietary product of the invention.

An “effective amount” for use in therapy of a condition is an amount sufficient to symptomatically relieve in a warm-blooded animal, particularly a human the symptoms of the condition or to slow the progression of the condition. In some aspects, an “effective amount” is an amount sufficient to reduce asparagine levels in the blood serum of a subject and/or to delay or inhibit metastasis.

The term “therapeutically effective amount” encompasses the amount of a compound or composition that, when administered, is sufficient to prevent development of, or alleviate to some extent, metastasis in a subject. The term “therapeutically effective amount” also encompasses the amount of a compound or composition that is sufficient to elicit the biological or medical response of a cell, tissue, organ, system, animal or human, which is being sought by a researcher, medical doctor or clinician. An appropriate “effective” amount in any individual case may be determined by one of ordinary skill in the art using routine experimentation. It will be understood that the specific dose level and frequency of administration for any particular patient may be varied and will depend upon a variety of factors, including the activity of the specific compound employed; the bioavailability, metabolic stability, rate of excretion and length of action of that compound; the mode and time of administration of the compound; the age, body weight, general health, sex and diet of the patient; and the severity of the particular condition being treated.

The terms “treat”, “treating” and “treatment” encompass alleviating or abrogating a condition, disorder or disease, or one or more of the symptoms associated with the condition, disorder or disease, and encompass alleviating or eradicating the cause(s) of the condition, disorder or disease itself. In certain embodiments, the terms “treat”, “treating”, and “treatment” refer to administration of a compound, a pharmaceutical composition or a pharmaceutical dosage form to a subject for the purpose of alleviating, abrogating or preventing a condition, disorder or disease, or symptom(s) associated therewith, or cause(s) thereof.

Suitably, the pharmaceutical composition of the invention may further comprise a therapeutic agent selected from: an inhibitor of cancer cell growth, a radiotherapeutic agent, an anti-metastic agent, an immune checkpoint inhibitor, a chemotherapeutic agent, an inhibitor of amino acid metabolism/turnover/inter-conversion, an inhibitor of non-essential amino acid biosynthesis, an inhibitor of amino acid transport, an enzyme or drug which promotes amino acid degradation or substance which sequesters amino acid(s).

### Cancer Therapy

In one aspect, the present invention provides a dietary product of the invention or produced in accordance with a process of the invention or a pharmaceutical composition of the invention for use in a medicament.

For example, the present invention provides a dietary product of the invention or produced in accordance with a process of the invention or a pharmaceutical composition of the invention for use in delaying or inhibiting metastasis.

As used herein, the term “metastasis” refers to the growth of a cancerous tumour in an organ or body part, which is not directly connected to the organ of the original cancerous tumour. Metastasis can also be defined as several steps of a process, such as the departure of cancer cells from an original tumour site, and migration and/or invasion of cancer cells to other parts of the body. Therefore, the present invention contemplates delaying or inhibiting further growth of one or more cancerous tumours in an organ or body part which is not directly connected to the organ of the original cancerous tumour and/or any steps in a process leading up to that growth.

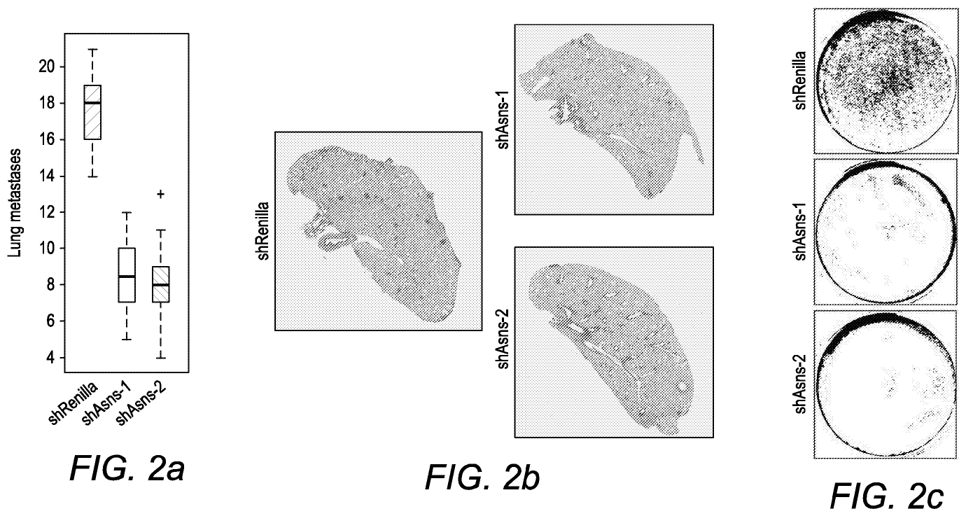

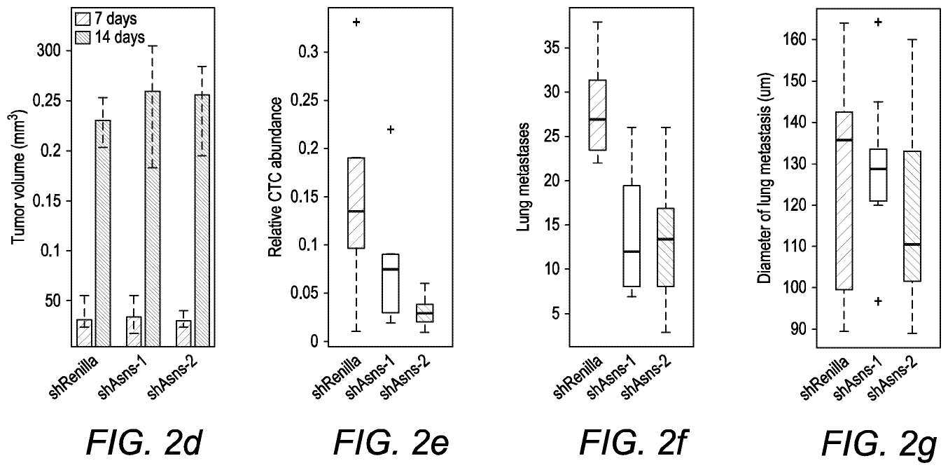

The present invention has surprisingly shown that by reducing asparagine levels (e.g. in the blood serum) of a subject with cancer, metastasis can be delayed or inhibited. In this regard, the present inventors have shown that silencing asparagine synthetase reduced metastatic potential both in vivo and invasive potential in vitro—see FIG. 2 for example. Treatment with L-asparaginase to reduce extracellular asparagine levels (i.e. bioavailability of asparagine) was shown to result in a significant reduction in metastatic burden in 4T1 cells injected NSG mice and furthermore depleting asparagine from the diet was also shown to lead to a reduction of metastatic burden in animals.

Accordingly, the present invention provides a medicament for use in delaying or inhibiting metastasis in a subject with cancer, wherein said medicament reduces extracellular asparagine levels in the blood of the subject with cancer. In a further aspect, the present invention provides the use of a compound or composition in the manufacture of a medicament for delaying or inhibiting metastasis in a subject with cancer, wherein the compound or composition reduces extracellular asparagine levels in the blood of the subject.

The present invention also provides a method of treating cancer in a subject, comprising administering a therapeutically effective amount of a medicament to said subject, wherein said medicament reduces extracellular asparagine levels in the blood of the subject with cancer.

A person of ordinary skill in the art is readily aware of various medicaments, compounds and compositions which can reduce asparagine levels in the blood of a subject with cancer.

These include asparagine synthetase inhibitors, L-asparaginase as well as dietary products as detailed herein.

Asparagine synthetase inhibitors are known in the art and include: guanidinosuccinic acid; oxaloacetic acid; L-cysteinesulfinic acid; diethyl aminomalonate; dipeptides containing L-aspartic acid (L-aspartylglycine, L-aspartyl-L-leucine, L-aspartyl-L-phenylalanine, L-aspartyl-L-proline, L-α-aspartyl-L-serine and L-α-aspartyl-L-valine); N-o-nitrophenylsulfenyl-L-aspartic acid; N-o-nitrophenylsulfenyl-L-glutamine; S-adenosyl-L-methionine; L-homoserine-β-adenylate; ethacrynic acid; Mupirocin, phosmidosine, β-asparaginyladenylate, sulfonamide analogs of the βAspAMP intermediates. Any asparagine synthetase which is suitable for use in therapy may be used in the present invention.

For all aspects, exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, meduUoblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplasia syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. Suitably, the cancer may be selected from the group consisting of breast cancer, colon cancer, squamous head and neck cancer, renal clear cell cancer and endometrial cancer.

The dietary product may be substantially devoid of cysteine. Suitably, a diet substantially devoid of cysteine may have utility in cancers which avidly consume exogenous cysteine such as lung, colorectal and breast cancer. Suitably, a diet substantially devoid of cysteine may have utility in cancers where there is a downregulated expression of MTAP.

The dietary product may be substantially devoid of serine and/or glycine. Suitably, a diet substantially devoid of serine and/or glycine may have utility in cancers which rely avidly consume exogenous serine and/or glycine such as lung, colorectal and breast cancer, lymphoma, colorectal cancer, liver cancer, osteosarcoma and breast cancer.

The dietary product may be substantially devoid of arginine and/or tyrosine. Suitably, a diet substantially devoid of arginine may have utility in cancers such as colorectal cancer.

### Combination Therapy

The medicaments, compounds and compositions which reduce asparagine levels in the blood of a subject (such as dietary products or pharmaceutical compositions of the invention) may be used alone to provide a therapeutic effect (e.g. to delay or inhibit metastasis). Suitably, the dietary products or pharmaceutical compositions of the invention may also be used in combination with one or more inhibitor(s) of cancer cell growth, anti-metastic agent(s), immune checkpoint inhibitor(s), chemotherapeutic agent and/or radiotherapy.

Such chemotherapy may include one or more of the following categories of anti-cancer agents:

(i) antiproliferative/antineoplastic drugs and combinations thereof, such as alkylating agents (for example cis platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5 fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, irinotecan, mitoxantrone and camptothecin); bleomcin, dactinomycin, daunorubicin, doxorubicin, epirubicin, idarubicin, ara-C, paclitaxel (Taxol™), nabpaclitaxel, docetaxel, mithramycin, deoxyco-formycin, mitomycin-C, L-asparaginase, interferons (especially IFN-alpha), etoposide, teniposide, DNA-demethylating agents, (for example, azacitidine or decitabine); and histone de-acetylase (HDAC) inhibitors (for example vorinostat, MS-275, panobinostat, romidepsin, valproic acid, mocetinostat (MGCD0103) and pracinostat SB939);
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride; and navelbene, CPT-II, anastrazole, letrazole, capecitabine, reloxafme, cyclophosphamide, ifosamide, and droloxafine;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti erbB2 antibody trastuzumab [Herceptin™]. the anti-EGFR antibody panitumumab, the anti erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI 1033), afatinib, vandetanib, osimertinib and rociletinib) erbB2 tyrosine kinase inhibitors such as lapatinib) and antibodies to costimulatory molecules such as CTLA-4, 4-IBB and PD-I, or antibodies to cytokines (IL-I0, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors. IGF receptor, kinase inhibitors; aurora kinase inhibitors and cyclin dependent kinase inhibitors such as CDK2 and/or CDK4 inhibitors; CCR2, CCR4 or CCR6 antagonists; and RAF kinase inhibitors such as those described in WO2006043090, WO2009077766, WO2011092469 or WO2015075483.
(v) antiangiogenic agents such as those which inhibit the effects of vascular endothelial growth factor, [for example the anti-vascular endothelial cell growth factor antibody bevacizumab (Avastin™)]; thalidomide; lenalidomide; and for example, a VEGF receptor tyrosine kinase inhibitor such as vandetanib, vatalanib, sunitinib, axitinib and pazopanib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100; dendritic cell-based vaccines (such as Ad.p53 DC); toll-like receptor modulators for example TLR-7 or TLR-9 agonists; PD-1, PD-L1, PD-L2 and CTL4-A modulators (for example Nivolumab), antibodies and vaccines; other IDO inhibitors (such as indoximod); anti-PD-1 monoclonal antibodies (such as MK-3475 and nivolumab); anti-PDL1 monoclonal antibodies (such as MEDI-4736 and RG-7446); anti-PDL2 monoclonal antibodies; and anti-CTLA-4 antibodies (such as ipilumumab); and
(viii) cytotoxic agents for example fludaribine (fludara), cladribine, pentostatin (Nipent™);
(ix) targeted therapies, for example PI3K inhibitors, for example idelalisib and perifosine; SMAC (second mitochondria derived activator of caspases) mimetics, also known as Inhibitor of Apoptosis Proteins (IAP) antagonists (IAP antagonists). These agents act to suppress IAPs, for example XIAP, cIAP1 and cIAP2, and thereby re-establish cellular apoptotic pathways. Particular SMAC mimetics include Birinapant (TL32711, TetraLogic Pharmaceuticals), LCL161 (Novartis), AEG40730 (Aegera Therapeutics), SM-164 (University of Michigan), LBW242 (Novartis), ML101 (Sanford-Burnham Medical Research Institute), AT-406 (Ascenta Therapeutics/University of Michigan), GDC-0917 (Genentech), AEG35156 (Aegera Therapeutic), and HGS1029 (Human Genome Sciences); and agents which target ubiquitin proteasome system (UPS), for example, bortezomib, carfilzomib, marizomib (NPI-0052), and MLN9708; and
(xii) chimeric antigen receptors, anticancer vaccines and arginase inhibitors.

The term “immune checkpoint” refers to a group of molecules on the cell surface of CD4+ and/or CD8+ T cells that fine-tune immune responses by down-modulating or inhibiting an anti-tumour immune response. Immune checkpoint proteins are well known in the art and include, without limitation, CTLA-4, PD-1, VISTA, B7-H2, B7-H3, PD-L1, B7-H4, B7-H6, 2B4, ICOS, HVEM, PD-L2, CD 160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein.

“Immune checkpoint inhibitors” refers to agents that inhibit immune checkpoint nucleic acids and/or proteins. Inhibition of one or more immune checkpoints can block or otherwise neutralize inhibitory signalling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc., that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signalling and upregulate an immune response. Alternatively, agents can indirectly block the interaction between one or more immune checkpoint proteins and its natural receptor(s) to prevent inhibitory signalling and upregulate an immune response. For example, a soluble version of an immune checkpoint protein ligand such as a stabilized extracellular domain can binding to its receptor to indirectly reduce the effective concentration of the receptor to bind to an appropriate ligand. Suitably, anti-PD-1 antibodies, anti-PD-LI antibodies, and anti-CTLA-4 antibodies, either alone or in combination, may be used to inhibit immune checkpoints.

The term “anti-metastatic agent” means a substance that inhibits, reduces or decreases metastasis of cancer cells. Anti-metastatic agents include, substances that inhibit or reduce angiogenesis, tissue factor activity, factor VIIa activity, or tissue factor/factor VIIa complex activity. Examples include VEG inhibitors, anti-VEGF antibodies (i.e. bevacizumab, AVASTIN), non-anticoagulant heparins, low molecular weight heparins (LMWH, such as LOVENOX), anti-tissue factor antibodies, CRA-5 (anti-fVIIa), BMS262084, TTP889, protein C, APC (Drotrecogin), sTM, Idraparinux, DX9065a, BAY59-7939, LY-51.7717, BMS-562247, DU-176b, otamixaban, razaxaban and NAP proteins.

Suitably, the composition of the present invention may be used in combination with one or more therapeutic enzymes which deplete amino acids. Such therapeutic enzymes may be correlated with the composition of the present invention. For example, for compositions which are substantially devoid of arginine, a therapeutic enzyme such as arginase may be used.

In addition, or in the alternative, the composition of the present invention may be used in combination with one or more compounds involved in the inhibition of de novo synthesis of amino acids. Such compounds may be correlated with the composition of the present invention. For example, the dietary product substantially devoid of asparagine may be used in combination with an asparagine synthetase inhibitor.

The therapeutic agent used in the present methods can be a single agent or a combination of agents. Preferred combinations will include agents that have different mechanisms of action.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In one aspect, the medicament, compound or composition (i.e. agent) which reduces asparagine levels in the blood is administered in a dosing regimen to obtain a desired therapeutic endpoint in terms of reduction of serum levels of asparagine or reduction of EMT phenotype and subsequently a therapeutic agent selected from: an inhibitor of cancer cell growth, an anti-metastatic agent, an immune checkpoint inhibitor, a radiotherapeutic agent and a chemotherapeutic agent is administered to the subject until a desired therapeutic endpoint in terms of cancer cell growth or proliferation is reached.

For example, the therapeutic endpoint of the dosing regimen may result in:
a reduction of asparagine in serum of at least 10% or at least 20% or at least 25% or at least 30% or at least 40% or at least 45% or at least 50%;
the asparagine content (%) in the serum free amino acid pool being less than 0.4% or less than 0.38% or less than 0.36% or less than 0.32% or less than 0.3%;
a reduction in at least one or more of: twist, e-cadherin and snail (e.g. in at least two or all three) in a tumour cell population of at least 20% or at least 30% or at least 40% or at least 50% or at least 60% or at least 70% or at least 80% or at least 90%.

The term “administered in combination with” and grammatical equivalents or the like, as used herein, are meant to encompass administration of the selected therapeutic agents to a single patient, and are intended to include treatment regimens in which the agents are administered by the same or different route of administration or at the same or different times. In some embodiments, the compounds described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present.

The agents disclosed herein may be administered by any route, including intradermally, subcutaneously, orally, intraarterially or intravenously.

In some embodiments in which a combination treatment is used, the amount of the dietary product or pharmaceutical composition of the invention and the amount of the other pharmaceutically active agent(s) are, when combined, therapeutically effective to treat a targeted disorder in the patient. In this context, the combined amounts are a “therapeutically effective amount” if they are, when combined, sufficient to reduce or completely alleviate symptoms or other detrimental effects of the disorder cure the disorder reverse, completely stop, or slow the progress of the disorder; delay or reduce metastasis or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

According to a further aspect of the invention, there is provided a dietary product or pharmaceutical composition of the invention as defined hereinbefore and an additional anti-cancer agent as defined hereinbefore, for use in the conjoint treatment of cancer.

According to a further aspect of the invention, there is provided a method of treatment of a human or animal subject suffering from a cancer comprising administering to the subject a therapeutically effective amount of a dietary product or pharmaceutical composition of the invention, simultaneously, sequentially or separately with an additional anti-cancer agent as defined hereinbefore.

According to a further aspect of the invention, there is provided a dietary product or pharmaceutical composition of the invention for use simultaneously, sequentially or separately with an additional anti-cancer agent as defined hereinbefore, in the treatment of a cancer.

The dietary product or pharmaceutical composition of the invention may also be used be used in combination with radiotherapy. Suitable radiotherapy treatments include, for example X-ray therapy, proton beam therapy or electron beam therapies. Radiotherapy may also encompass the use of radionuclide agents, for example 1311, 32P, 90Y, 89Sr, 153Sm or 223Ra. Such radionuclide therapies are well known and commercially available.

According to a further aspect of the invention, there is provided a dietary product or pharmaceutical composition of the invention, or a pharmaceutically acceptable salt thereof as defined hereinbefore for use in the treatment of cancer conjointly with radiotherapy.

According to a further aspect of the invention, there is provided a method of treatment of a human or animal subject suffering from a cancer comprising administering to the subject a therapeutically effective amount of a dietary product or pharmaceutical composition of the invention, or a pharmaceutically acceptable salt thereof simultaneously, sequentially or separately with radiotherapy.

In one aspect, the dose of each chemotherapeutic agent (or total combined dose of chemotherapeutic agents) may be equivalent to at least 0.1 g/Kg body weight of patient per day, preferably at least 0.2 g/Kg per day or 0.3 g/Kg per day or 0.4 g/Kg per day or 0.5 g/Kg per day. Suitably, the dose of chemotherapeutic agent (or combined combinations of chemotherapeutic agents) may be equivalent to at least 1 g/Kg per day, preferably 2 g/Kg per day.

Further, in another aspect, the present invention provides a method of treating cancer in a subject comprising administering a synergistically effective combination of: a) a dietary product of the invention and b) a chemotherapeutic agent.

In certain embodiments, the diet substantially devoid of asparagine comprises or consists of a dietary product.

The concentration of a therapeutic agent to be administered in accordance with the invention will vary depending on several factors, including the dosage of the compound to be administered, the pharmacokinetic characteristics of the compound(s) employed, and the route of administration. The agent may be administered in a single dose or in repeat doses.

Treatments may be administered daily or more frequently depending upon a number of factors, including the overall health of a patient, and the formulation and route of administration of the selected compound(s).

Preferably, said cancer treatment further comprises administration of a therapeutically effective amount of said therapeutic agent. The term “therapeutically effective amount” as used herein, refer to an amount of at least one agent or compound being administered that is sufficient to treat or prevent the particular disease or condition. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

In certain embodiments, the diet is administered over a time period of at least 1 day, 2 days, 3 days, 4 days, 5 days, 6 days. 7 days, at least 2 weeks, 3 weeks, 4 weeks. 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, or until a therapeutic endpoint is observed.

Where the diet substantially devoid of asparagine comprises or consists of a dietary product, the dietary product is administered from one to ten times daily.

### Stratification

The present invention has surprisingly shown that elevated levels of asparagine synthetase in a subject's primary tumour strongly correlates with later metastic relapse and that elevated asparagine levels in the blood of a subject with cancer contributes to metastasis.

Accordingly, serum asparagine levels or asparagine synthetase expression (e.g. in the primary tumour) may be used as a biomarker to identify a patient or patient population having a tumour which is at increased risk of metastasis.

Hence, the present invention provides a method of identifying a subject with cancer having an increased likelihood of metastasis comprising:
a) determining the level of asparagine in a biological sample isolated from the subject;
b) comparing the level of asparagine in the biological sample to a control sample or to a predetermined reference level of asparagine,
   wherein an increased level of asparagine in the biological sample compared to the control sample or compared to the predetermined reference level is indicative of increased likelihood of metastasis. The method may further comprise administering a therapeutically effective amount of a medicament to said subject, wherein said medicament reduces extracellular asparagine levels in the blood of the subject with cancer.

The present invention also provides a method of identifying a subject having an increased likelihood of responsiveness or sensitivity to a cancer treatment when fed a diet substantially devoid of asparagine or administered with L-asparaginase comprising:
a) determining the level of asparagine in a biological sample isolated from the subject;
b) comparing the level of asparagine in the biological sample to a control sample or to a predetermined reference level of asparagine,
   wherein an increased level of asparagine in the biological sample compared to the control sample or compared to the predetermined reference level is indicative of responsiveness or sensitivity to said cancer treatment when combined with a diet substantially devoid of asparagine or administered with L-asparaginase.

The present invention also provides a method of determining the likelihood of metastatic relapse in a subject having a cancer comprising:
a) determining the level of asparagine synthetase in a biological sample isolated from the subject;
b) comparing the level of asparagine synthetase in the biological sample to a control sample or to a predetermined reference level of asparagine synthetase,
   wherein an increased level of asparagine synthetase in the biological sample compared to the control sample or compared to the predetermined reference level is indicative of an increased likelihood of metastatic relapse.

Such methods may further comprise administering a therapeutically effective amount of a medicament to said subject, wherein said medicament reduces extracellular asparagine levels in the blood of the subject with cancer.

As used herein, the term “biological sample” and “sample isolated from a subject” are used interchangeably to refer to tissues, cells and biological fluids isolated from a patient, as well as tissues, cells and fluids present within a patient. The sample may be a urine sample, a blood sample, a serum sample, a sputum sample, a faecal sample, a biopsy of body tissues, for example a biopsy of transplanted kidney tissue, a cerebro-spinal fluid sample, a semen sample or a smear sample. A preferred sample is serum or plasma.

As used herein “reference leve” or “control”, refers to a sample having a normal level of asparagine/asparagine synthetase expression, for example a sample from a healthy subject not having or suspected of having cancer or, with regard to asparagine synthetase expression a sample from a tissue of the same subject not affected by cancer. Alternatively, the reference level/predetermined level may be a level from a reference database, which may be used to generate a pre-determined cut off value, i.e. a diagnostic score that is statistically predictive of a symptom or disease or lack thereof or may be a pre-determined reference level based on a standard population sample.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1 94); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### Examples

Investigation utilised 4T1-T compared with other 41T clones to identify drivers of metastasis, which exert their effects after a tumour cell has entered the blood stream. A combination of differential expression and focused in vitro and in vivo RNAi screens revealed candidate drivers of metastasis discriminating these clones, which were then evaluated in gene expression datasets from breast cancer patients. Among these, Asparagine Synthetase (Asns) expression in a patient's primary tumour was most strongly correlated with later metastatic relapse. Silencing of Asns reduced both metastatic potential in vivo and invasive potential in vitro. Conversely, increasing the availability of extracellular asparagine increased the invasive potential of mouse and human breast cancer cells, and enforced Asns expression promoted metastasis. Decreasing asparagine availability in mice by treatment with L-asparaginase or even by dietary restriction strongly reduced metastasis from orthotopic tumours. Asparagine availability varies between tissues, potentially explaining selective effects on particular steps of tumor progression. Asparagine limitation reduced the production of proteins that promote the epithelial to mesenchymal transition, providing one potential mechanism for how the availability of a single amino acid could regulate metastatic progression.

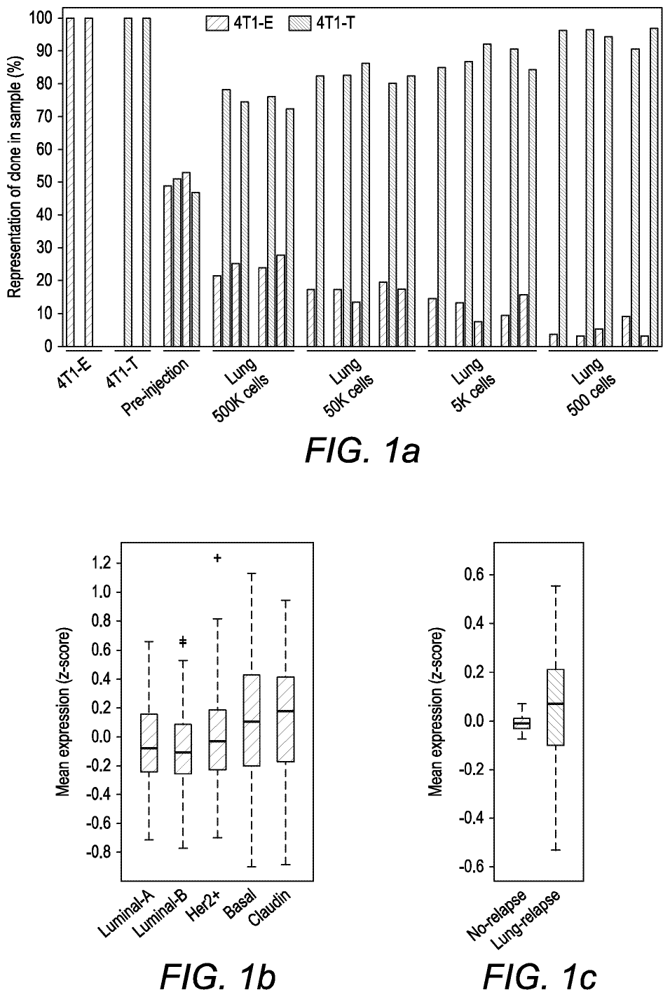

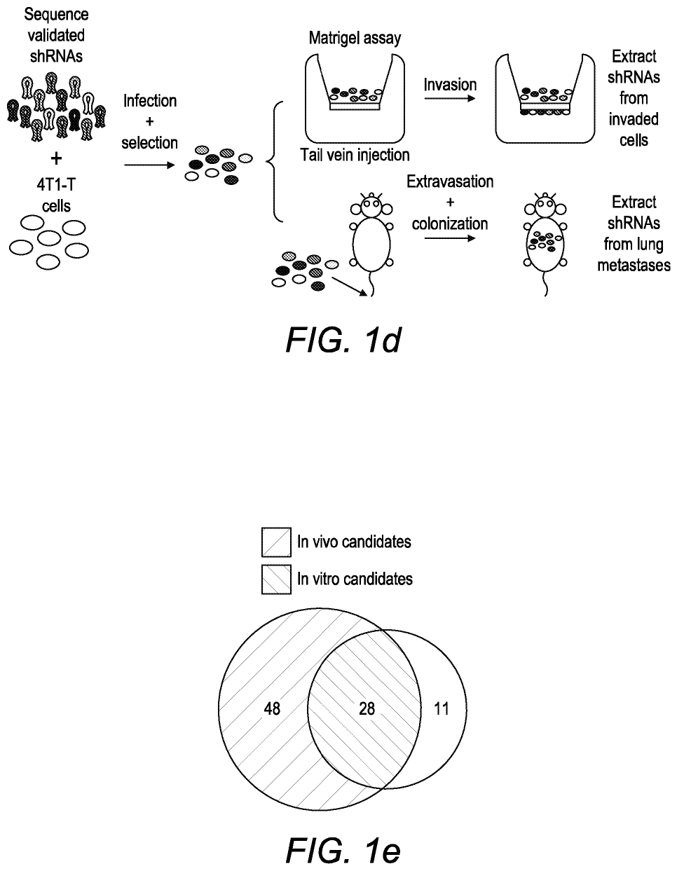

To validate the observation that 4T1-T had greater metastatic potential among CTC-proficient clones, the inventors combined equal numbers of 4T1-E and -T cells and introduced these directly into the bloodstream of immune compromised recipients (NOD-SCID-II2rg^{-/-} (NSG) mice) by tail-vein injection. Sequencing of the initial pool demonstrated that the two clones were present in equal abundance (FIG. 1a). However, when cells were harvested from the lung, clone T predominated, with its relative representation being inversely correlated with the total number of cells injected.

A differential gene expression analysis identified 192 genes with higher expression in 4T1-T as compared to 4T1-E (data not presented, fold-change >2, FDR<0.05). Their corresponding Gene Ontology terms were enriched for processes important for metastatic spread (data not presented, e.g. positive regulation of epithelial cell migration and regulation of locomotion) (Ashbumer et al 2000). A retrospective analysis of breast cancer patient data showed that genes within the set are more highly expressed in aggressive tumour subtypes (FIG. 1b, Basal and Claudin-low, ANOVA p-value <0.0001, Harrell et al 2012). Moreover, they are more highly expressed in the primary tumours of patients with later relapse to the bone, brain and lungs as compared to relapse-free survivors (FIG. 1c for lung, rank-sum p-value <0.01).

To determine whether this set of differentially expressed genes contained drivers of metastasis, the inventors carried out an RNAi screen with two parallel arms (FIG. 1d). In total, 26 pools of ˜50 shRNAs targeting the 192 genes (cumulatively accounting for ˜6 shRNAs per gene) were introduced into 4T1-T cells (Knott et al. 2014). Each pool was placed onto two separate 6 well matrigel invasion chambers or introduced into 5 replicate NSG mice by tail vein injection. After 24 hours, the cells which had invaded through the matrigel were collected and, after 7 days, lungs were harvested from the mice and perfused to exclude residual CTCs from the vasculature. Using high-throughput sequencing, the inventors identified shRNAs that were depleted in the invaded cell populations or lung metastases (empirical bayes moderated t-test FDR<0.05), presumably because they targeted genes important for these processes. Strong overlap was observed when the in vitro and in vivo candidates were compared (FIG. 1e, hypergeometric test p-value <0.0001). Genes were classified as candidates if at least two corresponding shRNAs depleted in each arm of the screen (data not presented).

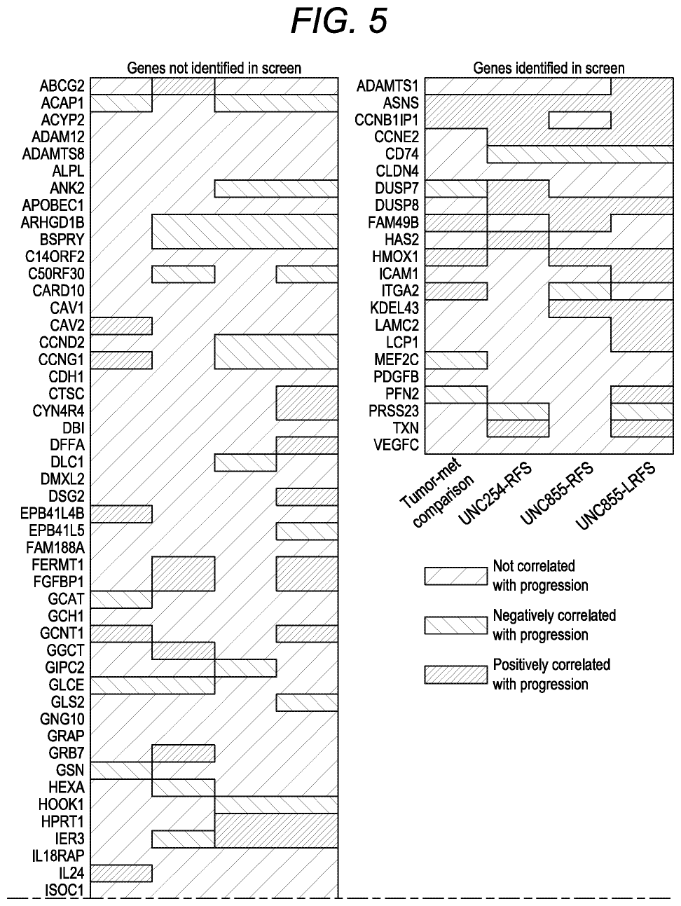

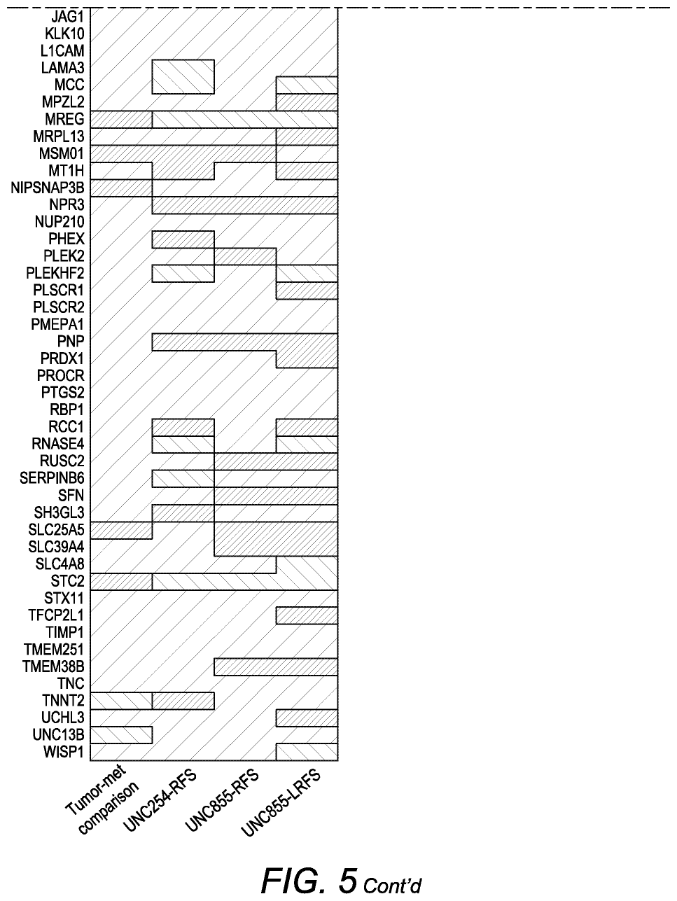

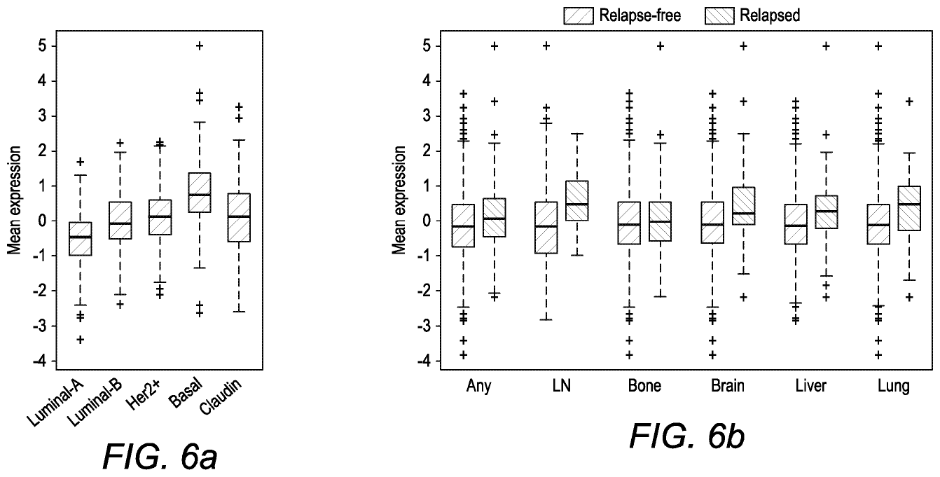

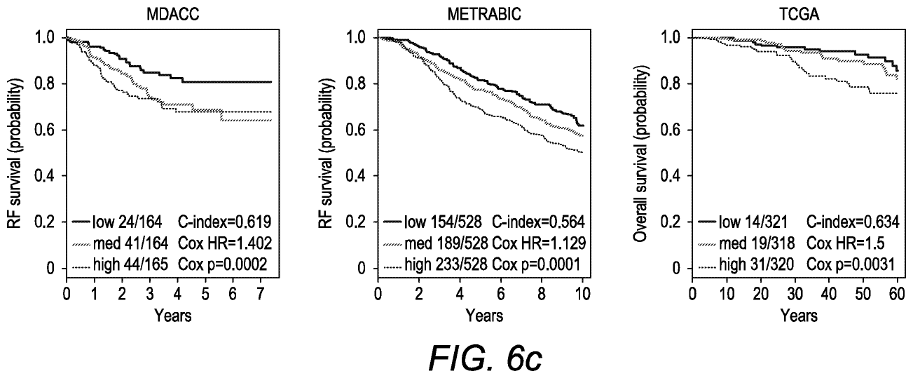

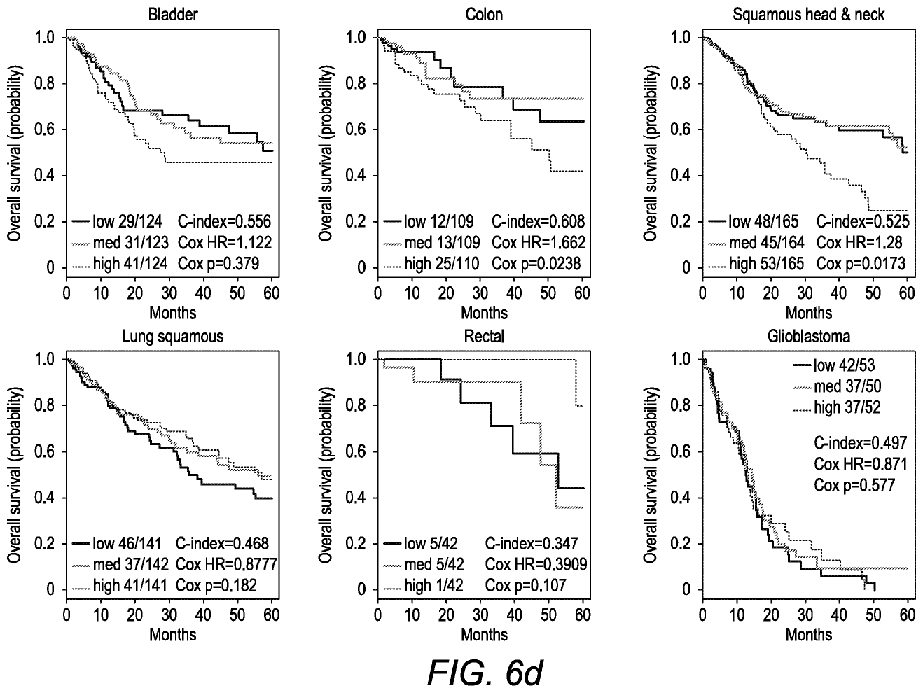

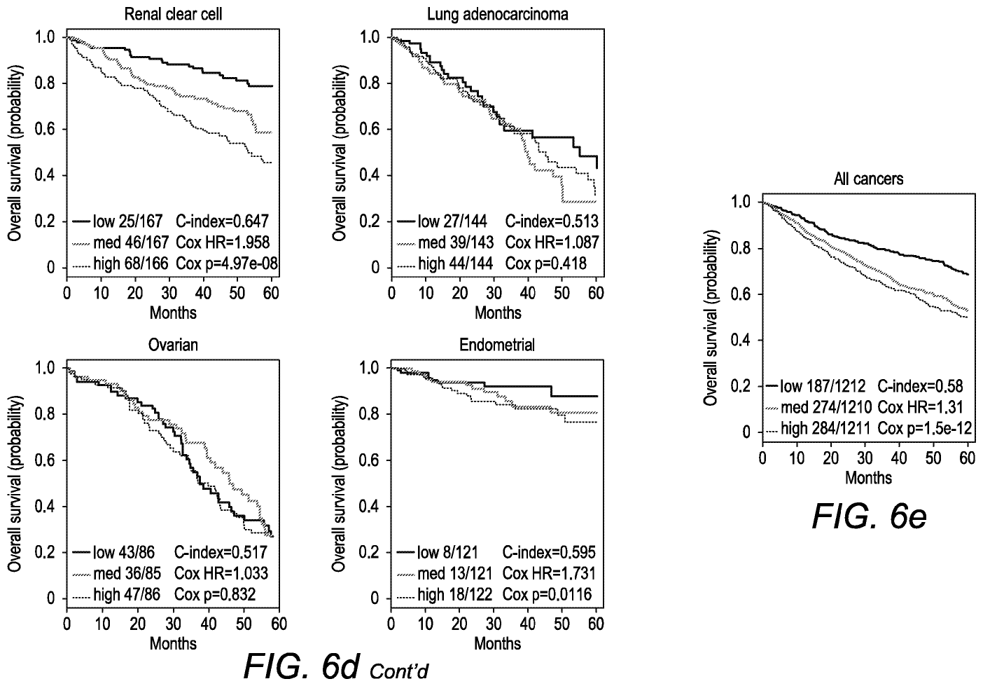

Of the candidate genes that scored in both the in vitro and in vivo assays, Asparagine Synthetase (Asns) had the most robust clinical evidence supporting its relevance to disease progression (FIG. 5). Expression levels of the human ortholog ASNS were predictive of general and lung-specific relapse in two breast cancer patient datasets (cox p-value <0.001). Also, when a small collection of matched tumour and lung metastases were transcriptionally profiled, ASNS was found to be more highly expressed in secondary lesions. ASNS is more highly expressed in aggressive tumour subtypes (Basal, Claudin-low and Her2+, FIG. 6a, ANOVA p-value <0.0001) and it is more highly expressed in patients with relapse to the lymph node, brain, liver and lungs as compared to relapse-free survivors (FIG. 6b, rank sum p-value <0.005). Subsequent analyses identified ASNS as predictive of survival in three additional breast cancer patient datasets (Extended Data FIG. 2c, cox p-value <0.001). In addition to breast, ASNS is negatively correlated with survival in 4 out of the 10 other solid tumors that are represented in the TCGA Pan-Cancer dataset (FIG. 6d, cox p-value <0.05). Finally, ASNS is also a globally predictive biomarker for solid tumours (FIG. 6e, cox p-value <0.001).

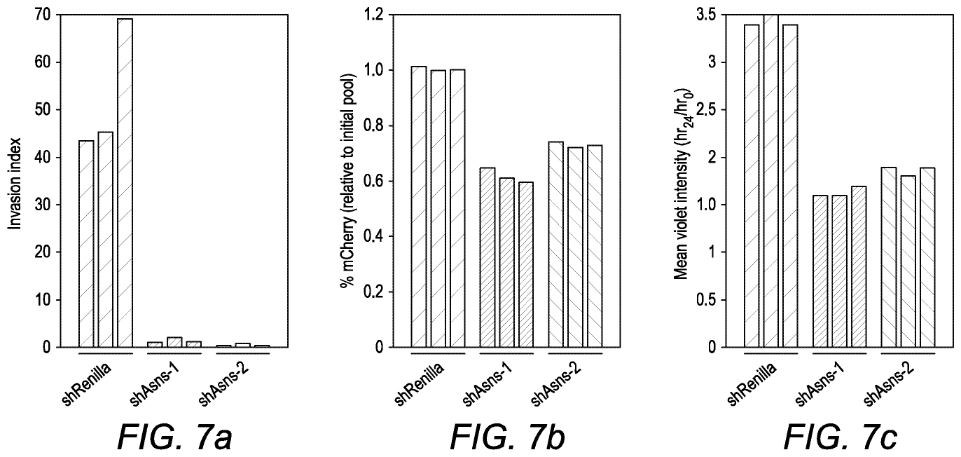

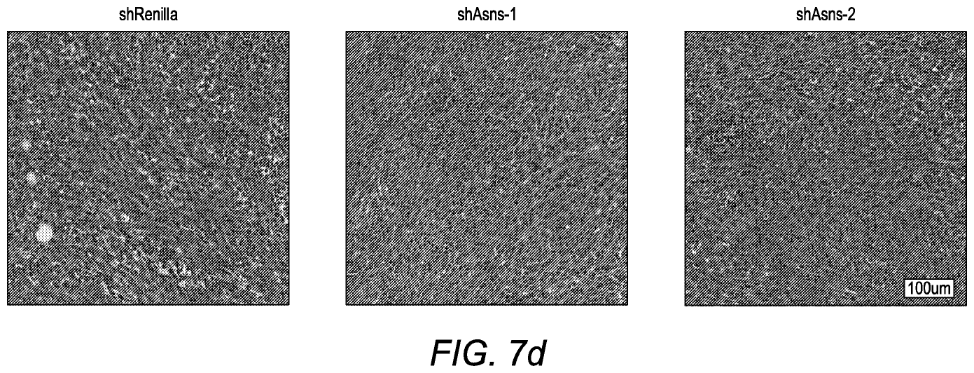

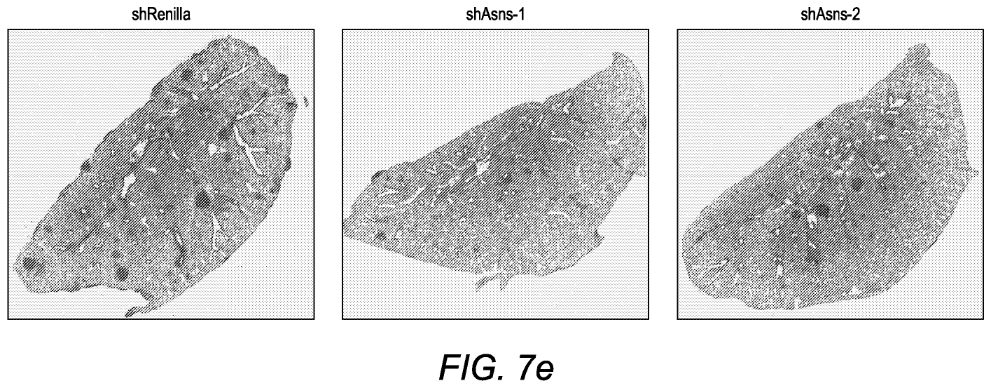

To validate Asns as a metastatic driver, the inventors infected 4T1-T cells with two shRNAs targeting Asns, or with a control shRNA targeting Renilla Luciferase and introduced these cells intravenously into NSG mice (data not presented). When lungs were assessed 9 days after injection, those animals receiving Asns-silenced cells showed significantly reduced metastatic burdens (FIG. 2a & FIG. b, rank-sum test p-value <0.001). Asns-silenced cells also showed poor invasion into matrigel (FIG. 2c & FIG. 7a). Silencing Asns did impact growth in vitro; however, this defect was minor compared to that observed in the invasion assay (Extended Data FIG. 7b & FIG. 7c).

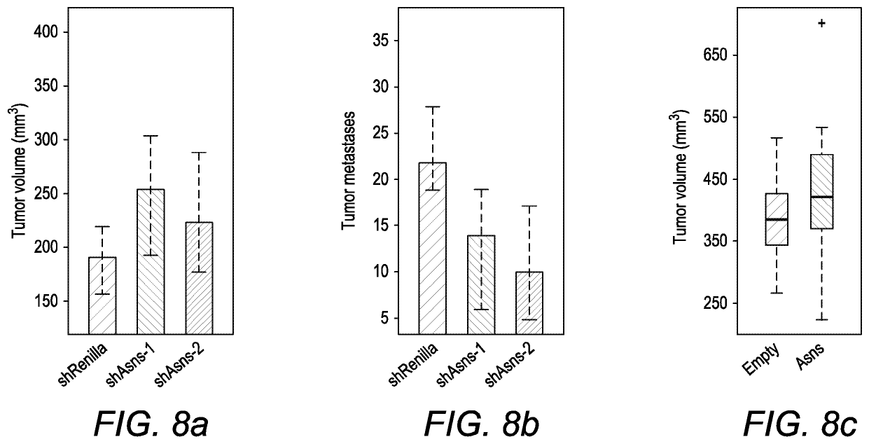

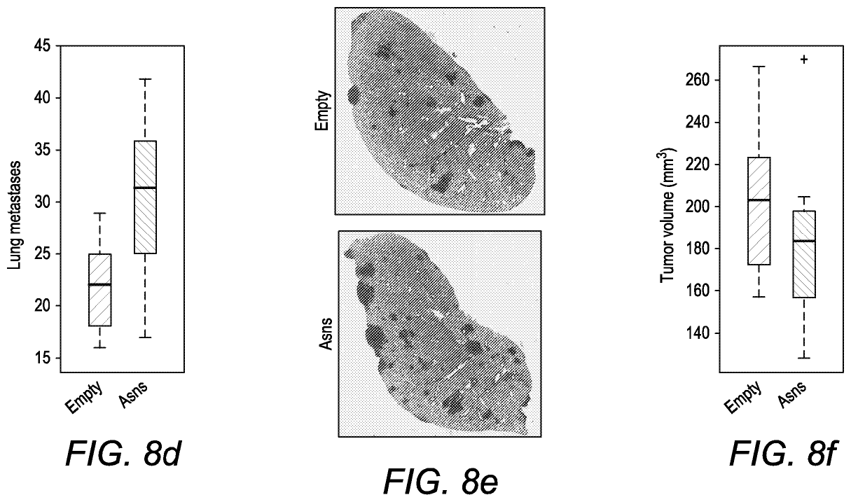

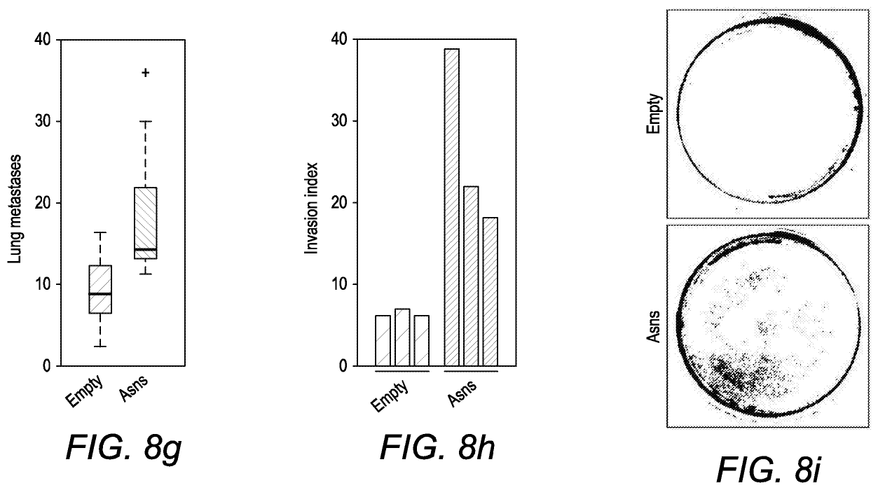

When Asns-silenced cells were injected orthotopically into the mammary fat pad, no significant change in primary tumour formation was observed (FIG. 2d & FIG. 7d). However, corresponding CTCs and lung metastases were reduced for tumours that were derived from Asns-silenced cells (FIG. 2e, FIG. 2f & FIG. 7e, rank-sum p-value <0.05 & <0.0002, respectively). The metastases that were initiated by silenced cells were noticeably smaller (FIG. 2g). Although this difference was deemed statistically insignificant, it does hint at growth being impacted at the metastatic site. Similar results were obtained with Asns-silenced parental 4T1 cells, indicating that Asns dependency is not a peculiarity of this single clonal line (FIG. 8a & FIG. 8b, rank-sum p-value <0.002). Enforced Asns expression in parental 4T1 populations did not affect primary tumour growth but corresponding metastases were significantly increased in number (FIG. 8c-8e, rank-sum p-value 0.02). Similar outcomes were observed upon enforced ASNS expression in a human breast cancer cell line, MDA-MB-231 (FIG. 8f-8i, rank-sum p-value <0.02 for ASNS-expression enforced vs. normal metastases).

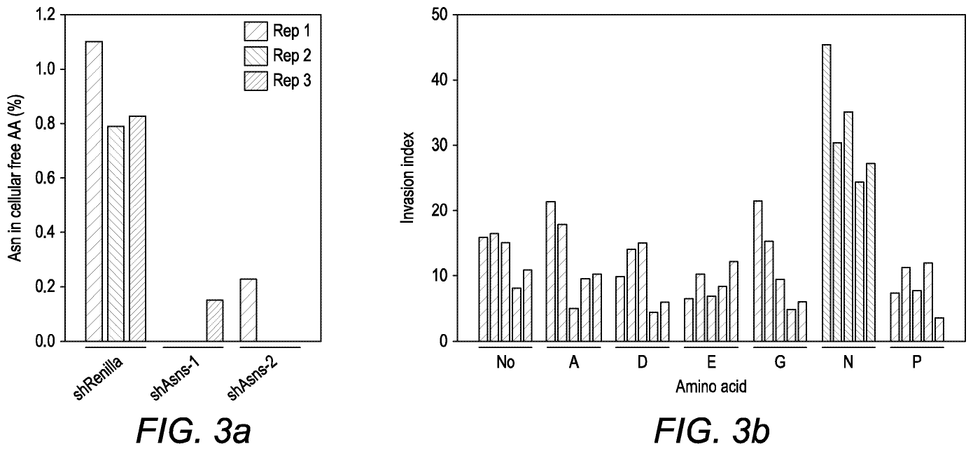

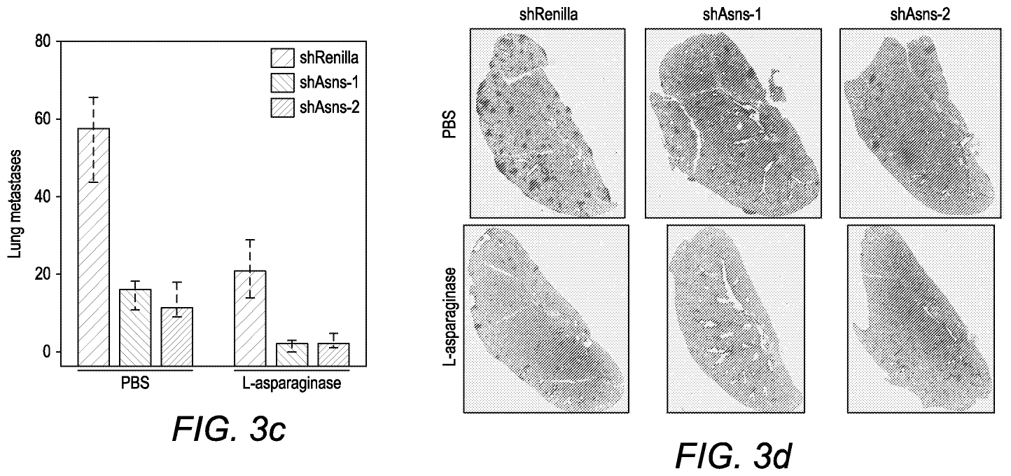

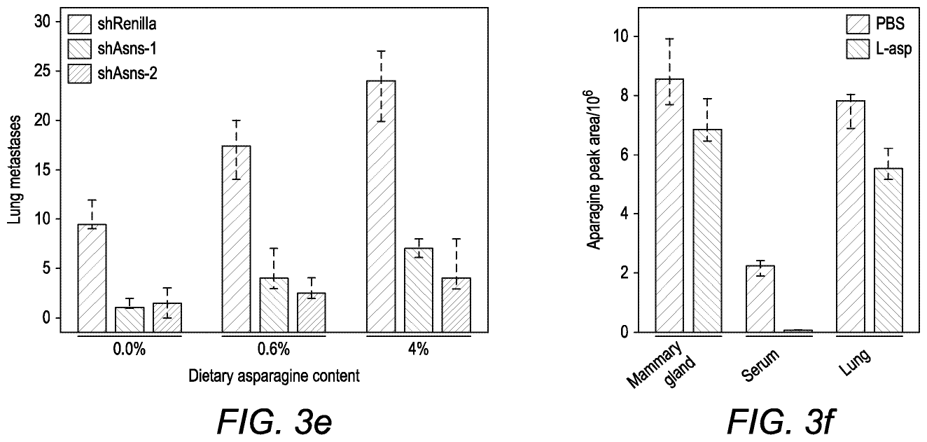

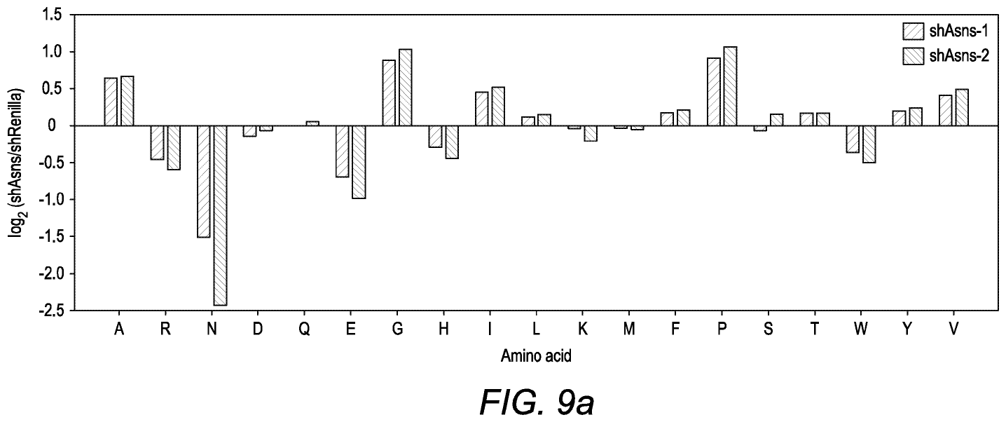

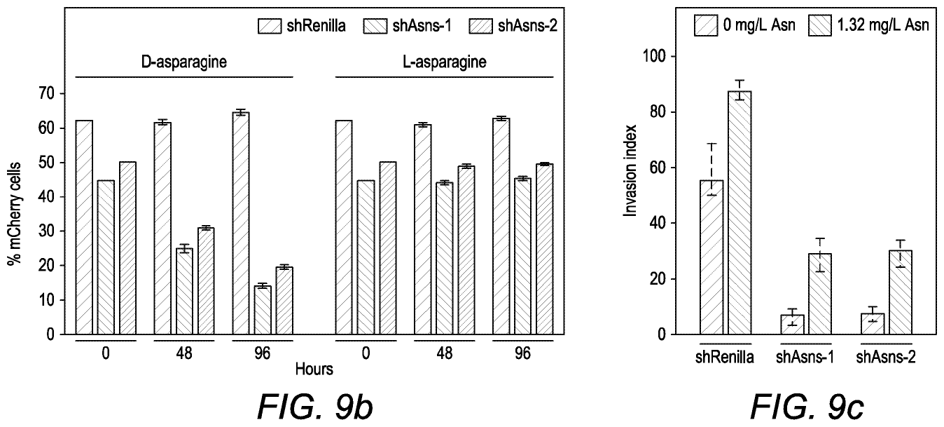

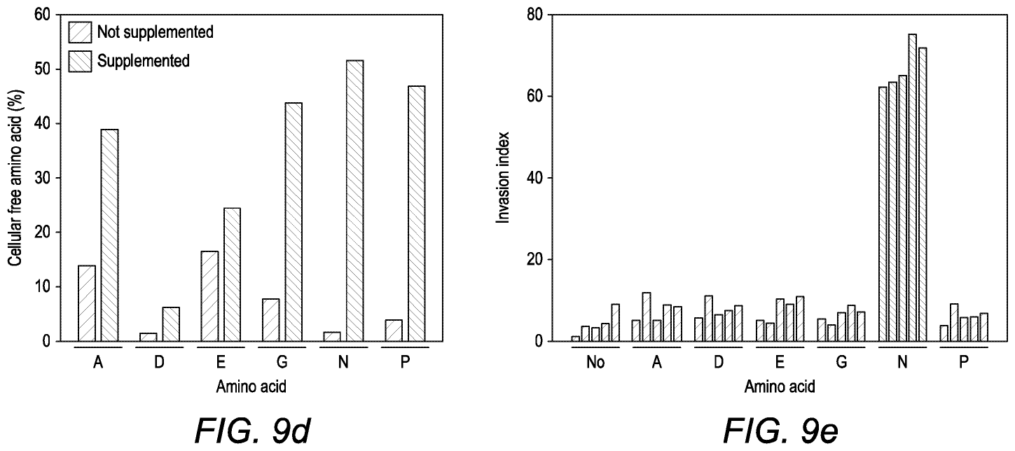

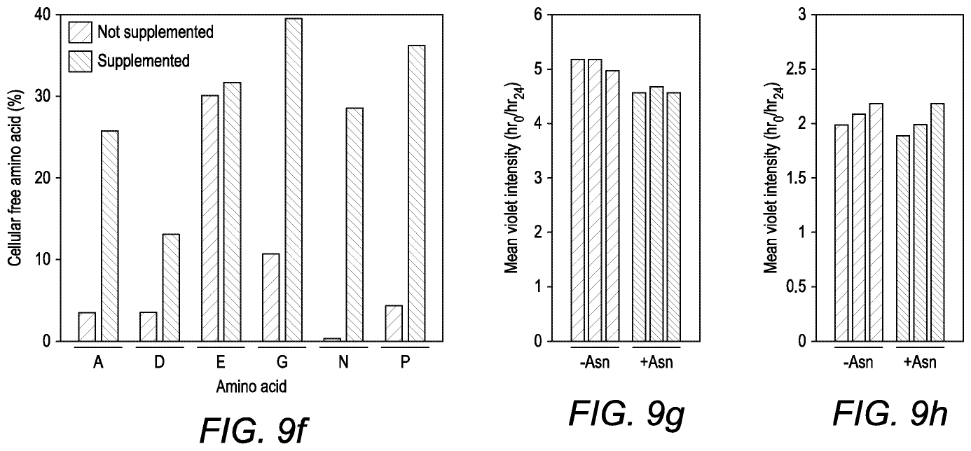

A significant reduction of intracellular free asparagine was detected upon silencing of Asns in 4T1-T cells (FIG. 3a & FIG. 9a, empirical bayes moderated t-test FDR<0.05). Furthermore, the capacities to invade and grow were increased in Asns-silenced cells when media was supplemented with asparagine (FIG. 9b & FIG. 9c). The inventors therefore asked whether the ability to promote invasion was unique among non-essential amino acids that were lacking in the DMEM culture media (alanine, aspartate, glutamic acid and proline). The inventors supplemented media separately with each of these amino acids, as well as with glycine as a negative control, and assayed the invasiveness of cells grown in each condition. HPLC confirmed that levels of uptake were similar for each of the amino acids, with the exception of aspartic and glutamic acid (FIG. 9d). 4T1 cells responded uniquely to asparagine supplementation, with an approximately 2-fold increase in invasiveness (FIG. 3b, rank-sum p-value <0.01). Similar outcomes were observed with MDA-MB-231 cells (FIG. 9e & FIG. 9f). Growth was not impacted by asparagine supplementation for either cell line (FIG. 9g & FIG. 9h, rank-sum p-value <0.01).

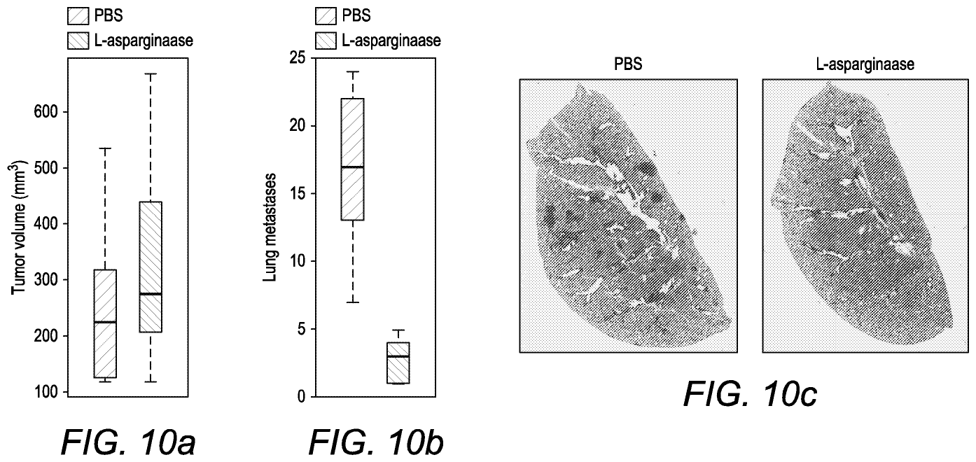

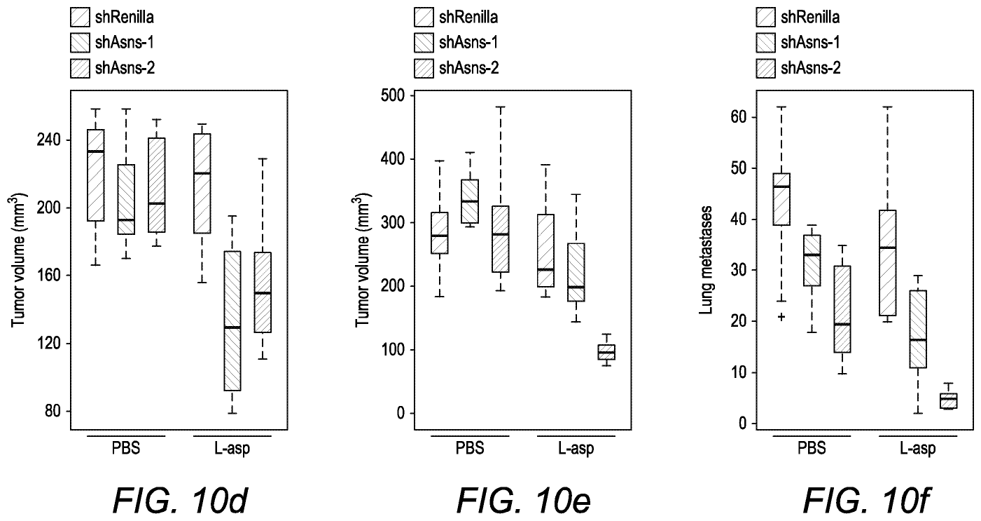

These in vitro assays indicated that invasion is influenced by asparagine availability, which is dictated by both biosynthetic capacity and the local environment. The inventors therefore asked whether metastasis could be influenced by a reduction in extracellular asparagine availability. Limiting the availability of asparagine to cancer cells is a strategy that is successfully employed to treat patients with acute lymphoblastic leukemia (ALL) (Richards et al. 1998, Richards et al. 2006), using a bacterial asparagine hydrolyzing enzyme, L-asparaginase, to reduce extracellular asparagine. L-asparaginase has proven ineffective for treating solid tumours (Tallal et al. 1970), in accord with results indicating that Asns expression is not a requirement for growth at the primary site. To determine if disease progression is impacted by systemic asparagine levels, the inventors orthotopically injected parental 4T1 cells into NSG mice and treated half of the animals with 60U L-asparaginase 5 times per week for 19 days, reducing serum asparagine to levels undetectable by HPLC (data not shown). While no significant difference in primary tumour volumes was detected, a significant reduction in metastatic burden was observed in L-asparagine treated mice (FIG. 10a-c, rank-sum p-value <0.0002).

ALL patients are typically classified as being positive or negative for the TELAML1 fusion gene. Those that are positive have high response rates when treated with chemotherapeutic cocktails containing L-asparaginase (Ramakers-van Woerden et al. 2000, Stams et al. 2005). TELAML1 negative patients, however, are more prone to resistance, and it is in these patients where high ASNS levels are observed after therapy, indicating that resistance may be achieved through biosynthetic production. In the present model, when Asns-silenced 4T1 cells were injected orthotopically into L-Asparagine treated mice, resultant metastases were nearly undetectable (FIG. 3c & FIG. d, rank-sum p-value <0.0005). In this case, a reduction of primary tumour volume was also observed (Extended Data FIG. 6d, rank-sum p-value <0.05). Similar results were obtained with ASNS-silenced MDA-MB-231 cells (FIG. 10e & FIG. 10f, rank-sum p-value <0.05).

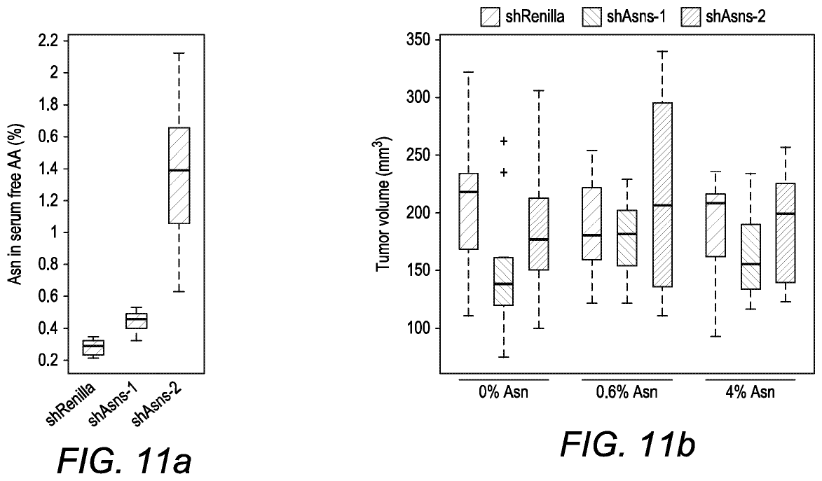

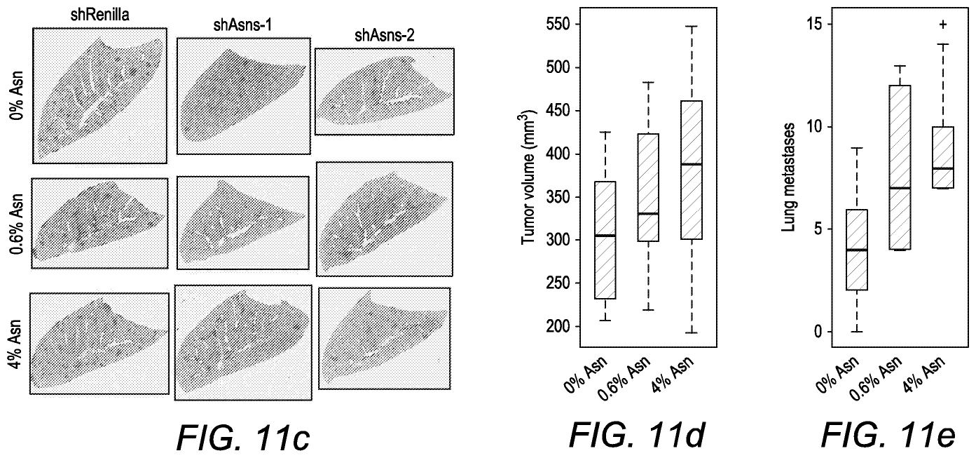

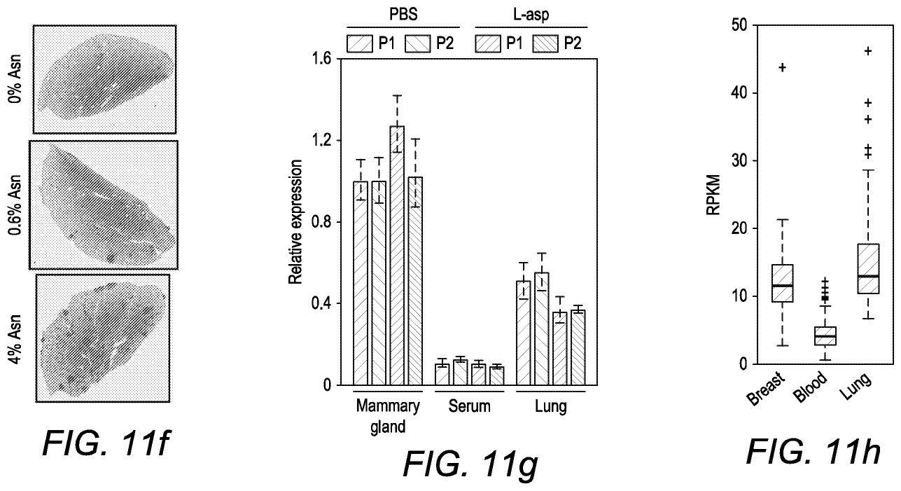

The availability of extracellular asparagine can be manipulated not only by treatment with L-asparaginase but also by depleting asparagine from the diet. shRNA-infected 4T1-T cells were orthotopically injected into mice that received either a control, low-asparagine or high-asparagine diet (0.6%, 0%, and 4%, respectively). HPLC confirmed that serum asparagine levels were significantly altered in concordance with dietary intake (FIG. 11a). Asparagine restriction did not impact primary tumour growth, regardless of Asns-expression status (FIG. 11b). In contrast, the metastatic burden was decreased in animals that were fed low-asparagine diets and increased in animals that were given high-asparagine diets regardless of Asns-expression status (FIG. 3e & FIG. 11c, rank-sum p-value <0.0005). Metastases were nearly undetectable in mice that were injected with Asns-silenced cells and fed a low asparagine diet. Similar results were obtained when parental 4T1 cells were orthotopically injected into animals fed these same asparagine-controlled diets (FIG. 11d-f, rank-sum p-value <0.05).

Metabolomic analyses of the mammary gland, serum and lungs by mass spectrometry revealed that, under normal physiological conditions, asparagine levels are highest in the mammary gland and lowest in the serum (FIG. 3f, rank-sum p-value <0.0005). Asparagine was nearly undetectable in the serum of L-asparaginase treated animals. qPCR analysis determined that asparagine abundance correlated with Asns expression levels in those tissues (FIG. 11g, rank-sum p-value <0.05). High asparagine availability in the mammary gland might buffer the impact of Asns-silencing or changes in global asparagine levels, so that tumour growth rates are maintained. However, low levels in the serum make cells susceptible to these changes. Finally, intermediate levels in the lung may explain the smaller metastases that result from Asns silencing. ASNS expression levels follow a similar patter across human tissues, however here expression in the lung is slightly higher than in the breast (FIG. 11h).

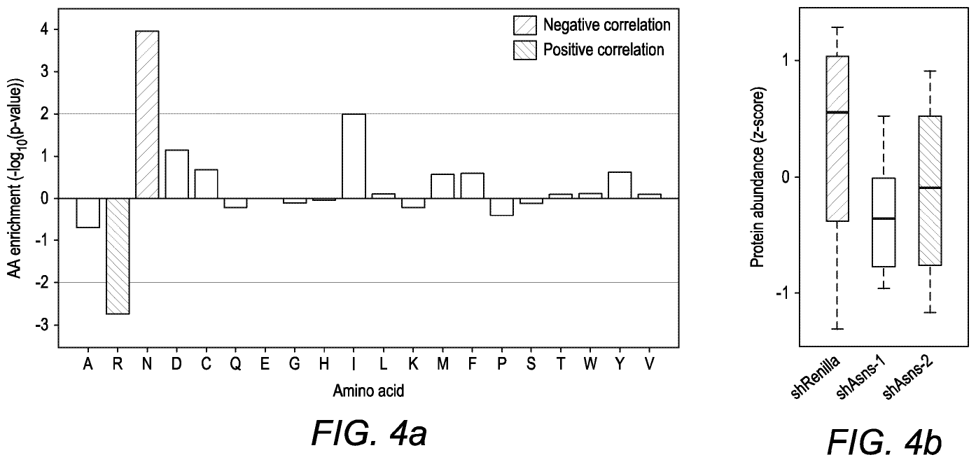

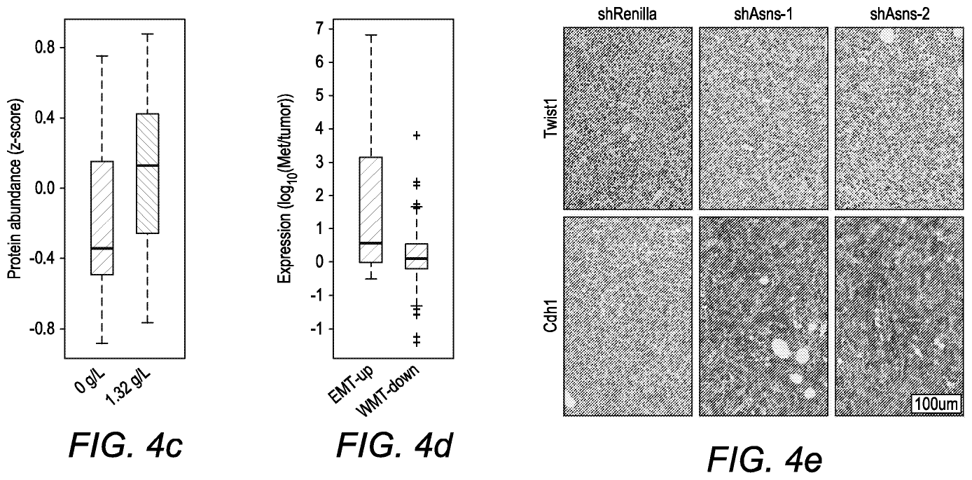

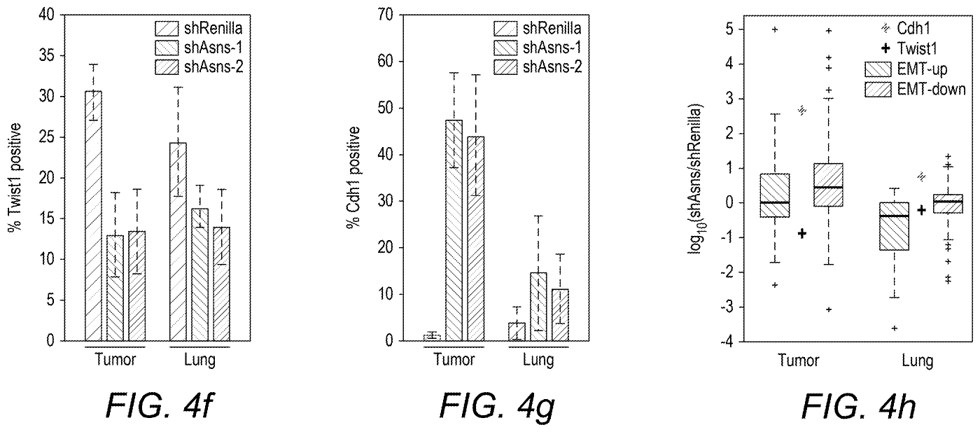

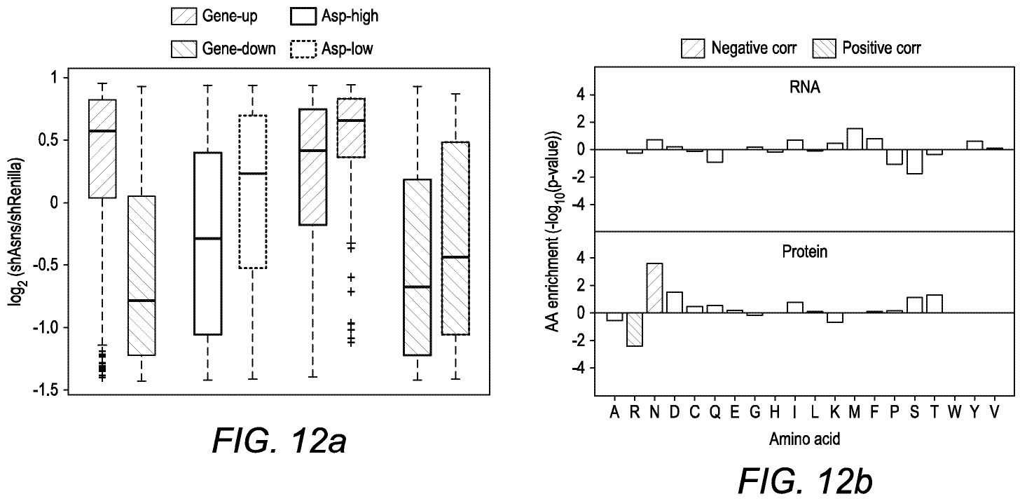

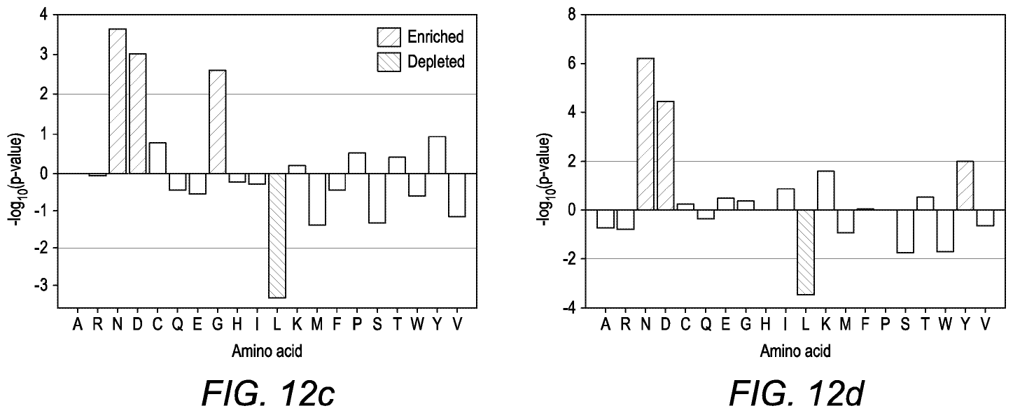

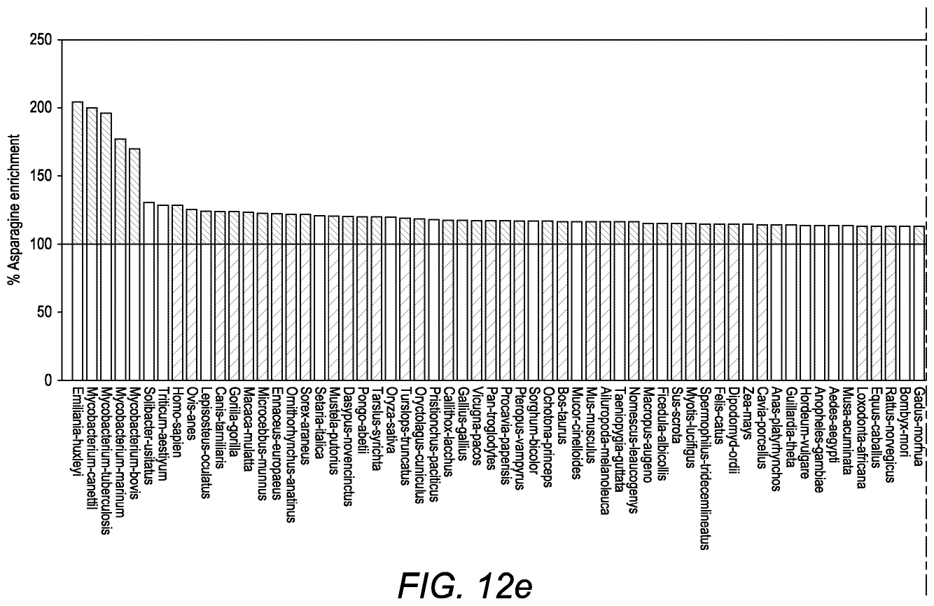

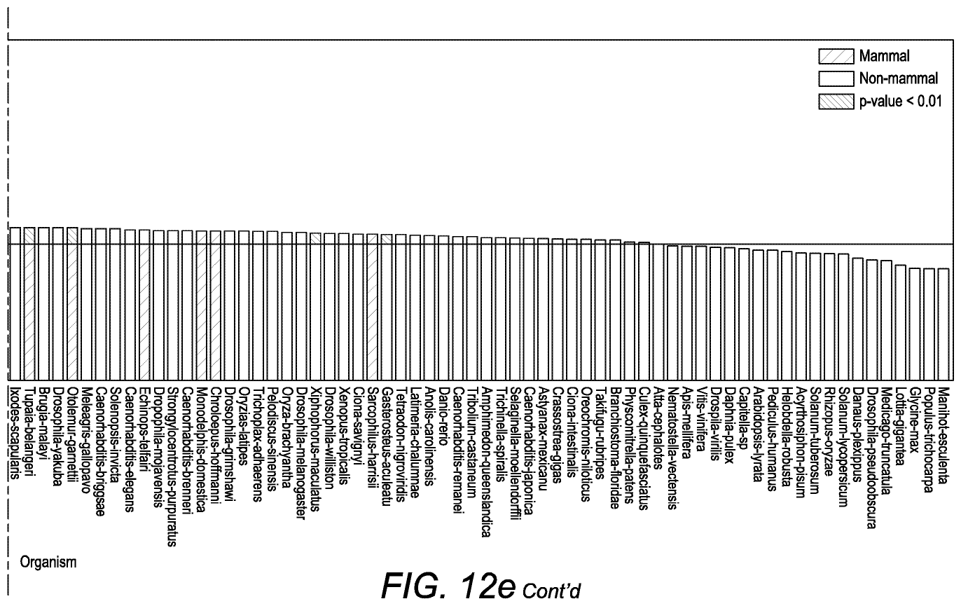

To understand the underlying mechanism by which asparagine availability might impact invasion and metastasis, the inventors examined expression changes induced by Asns silencing, both at the RNA and protein level. RNA measurements were the strongest predictor of protein-level changes (FIG. 12a, rank-sum p-value <0.001). In accordance with a previous report of translational pausing at asparagine residues in L-asparaginase treated cells, the inventors also found asparagine content to be predictive of corresponding protein-level changes (FIG. 12a, rank-sum p-value <0.001, Loayza-Puch et al. 2016). This was true for both un-normalized differences and for changes that varied from what was predicted by corresponding RNA measurements (FIG. 12b & FIG. 4a, rank-sum p-value <0.001).

Among the asparagine-enriched proteins that were decreased in expression upon Asns-silencing, the inventors found an over representation of genes whose human orthologues were previously identified as up-regulated when epithelial-to mesenchymal transition (EMT) was induced (Taube et al. 2010) (FIG. 4a, FIG. 4b and FIG. 12c, EMT-up proteins, hypergeometric p-value <0.005). Depleted proteins have an 18% higher asparagine content than the analyzed protein pool, while EMT-up proteins are 20% higher in content. These same proteins were increased in expression when the media of parental 4T1 cells was supplemented with asparagine (FIG. 4c, sign-rank p-value <0.05). The human orthologues are also enriched in asparagine (FIG. 12d, rank-sum p-value <0.001), with the second most enriched amino acid being aspartate, the substrate for intracellular asparagine biosynthesis. Further, asparagine enrichment is a globally conserved property of EMT-up proteins (FIG. 12e, sign-rank p-value <1.0×10⁻¹³) with the levels being highest in mammals (rank-sum p-value <9.0×10⁻⁹).

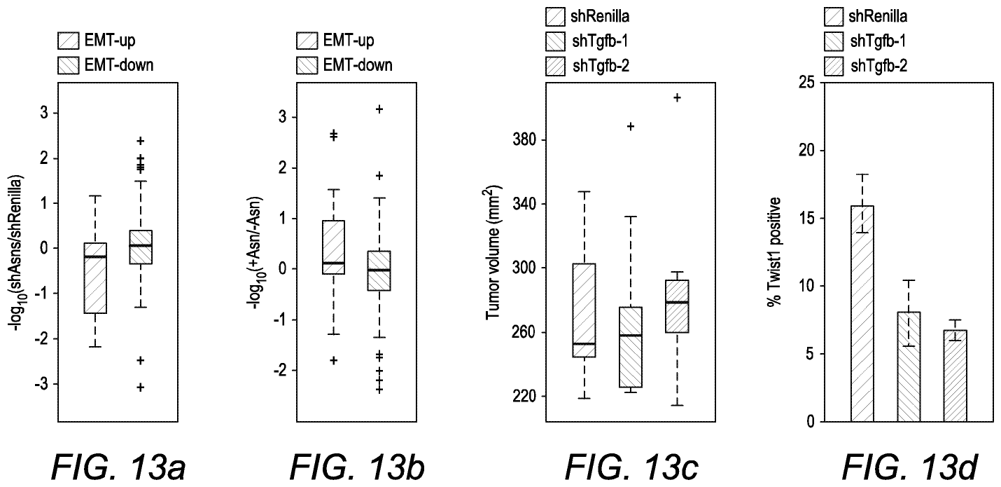

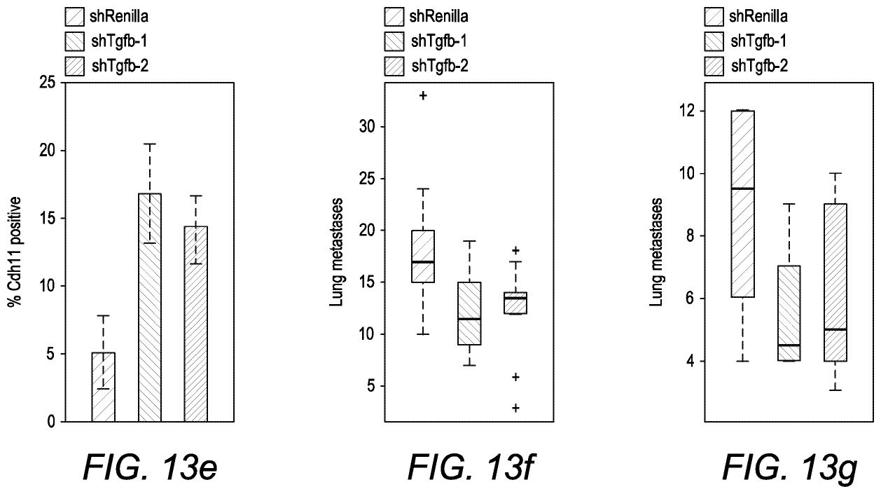

EMT-up genes were also down-regulated at the transcriptional level in Asns-silenced cells (FIG. 13a, sign-rank p-value <0.001). In addition, the mRNA levels of two prototypical EMT markers (Twist1 and E-Cadherin) were altered to indicate a perturbed EMT program (DESeq, FDR<0.05). EMT-up genes were also increased in their mRNA levels when parental 4T1 cells were grown in asparagine supplemented media (FIG. 13b, sign-rank p-value <0.005). A reanalysis of existing data also showed reduced expression of EMT-up genes when ATF4, which regulates ASNS transcription, was deleted in haploid cells and liver cells from L-asparaginase treated ATF4 knockout mice were more perturbed in their EMT program than were similarly treated WT mice. Considered together, these data suggested that asparagine bioavailability might impact metastasis, at least in part, through regulation of EMT.

EMT has been strongly implicated in metastasis but the extent to which it is important is unknown. When gene expression profiles of matched parental 4T1 tumours and lung metastases were compared, EMT-up genes were found to be significantly increased in secondary lesions (FIG. 4d, sign-rank p-value <0.001). In contrast, genes that were down-regulated when EMT was induced (EMT-down genes) were unchanged. To functionally validate a role for EMT in this model, the inventors orthotopically injected 4T1-T cells in which the inventors had silenced expression of Tgf-ß, a key driver of EMT. Growth at the orthotopic site was not affected by Tgf-ß silencing (FIG. 13c). However, IHC of tumour sections revealed that Twist1 and E-cadherin (two prototypical EMT markers) were altered in Tgf-ß-silenced tumours (FIG. 13d & FIG. 13e, rank-sum p-value <0.01). Consistent with an important role for EMT, fewer metastases were observed in mice carrying tumors with impaired EMT (Extended Data 9f, rank-sum p-value <0.05). Tgf-ß-silenced cells also produced fewer metastases when they were intravenously injected, indicating that EMT plays a role in metastasis after tumour cells have entered the bloodstream (FIG. 13g, rank-sum p-value <0.05).

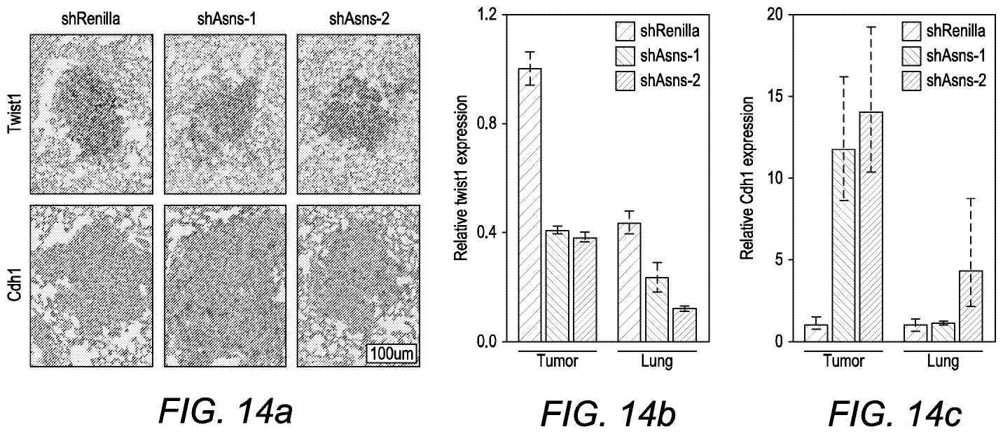

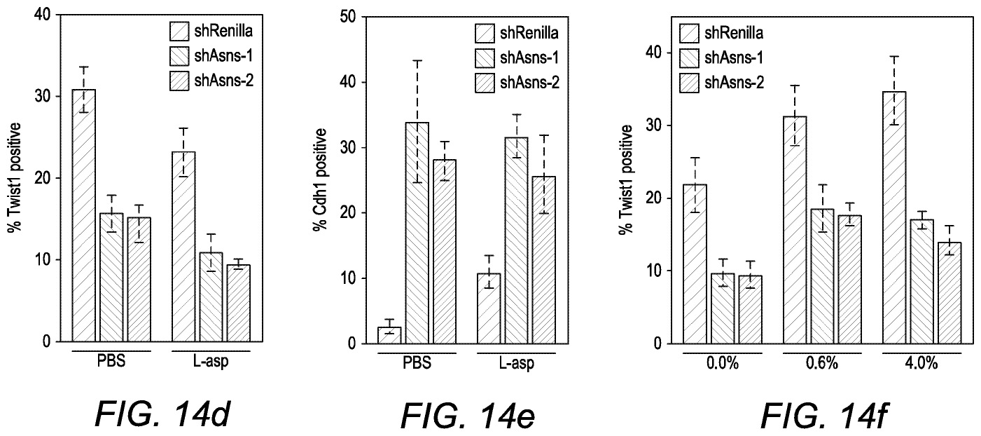

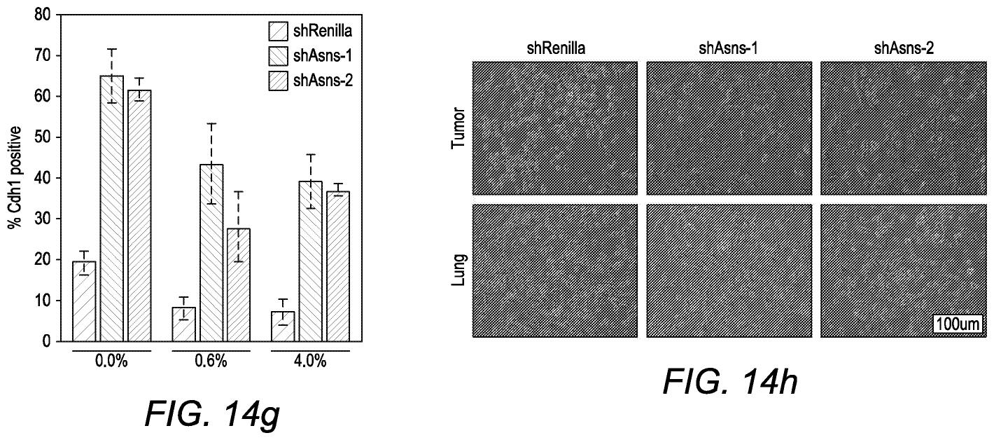

Though no morphological differences were detected in tumours derived from Asns-silenced cells, IHC staining for Twist and E-cadherin suggest that EMT is perturbed in these lesions, and this same pattern is observed in the corresponding metastases (FIG. 4e-g & FIG. 14a). When the malignant cells from these lesions were FACS-isolated and analyzed by qPCR, Twist1 and E-cadherin expression levels were found to be altered in a manner consistent with the staining (FIG. 14b & FIG. 14c, rank-sum p-value <0.05). Similar patterns were observed in the primary tumours of mice that had been treated with L-asparaginase (FIG. 14d & FIG. 14e). There, silencing had the greatest impact, but depletion of extracellular asparagine using L-asparaginase did elicit a significant expression change (rank-sum p-value <0.01). Dietary asparagine also impacted expression, with asparagine content correlating positively with the EMT-signature in the primary tumour (FIG. 14f & FIG. 14g9, rank-sum p-value <0.01).

To examine, more closely, the malignant cells within these lesions, the inventors isolated by 6-TG selection shRNA infected 4T1-T cells from tumours and lung metastases. Metastatic cells displayed a mesenchymal morphology (elongated and spindle shaped) regardless of Asns-expression status (FIG. 14h). However, Asns-silenced cells that were isolated from the primary tumour, displayed more epithelial morphology. In these cells, EMT-down genes are upregulated, and Twist and E-cadherin expression levels are also altered in a manner consistent with the EMT program being perturbed (FIG. 4h, sign-rank p-value <0.05 and DESeq FDR<0.05). EMT-up genes are down-regulated in Asns-silenced metastatic cells, and Twist and E-cadherin are altered in expression here as well (rank-sum p-value <0.001 and DESeq FDR<0.05).

Our model of breast tumour heterogeneity has strongly implicated asparagine bioavailability as a regulator of metastatic progression. This is also likely relevant in human cancers, as high ASNS expression is a marker of poor prognosis for many tumour types. One mechanism underlying our findings is likely a link between asparagine bioavailability and EMT, which can be observed in vitro and in vivo. In our breast cancer model, the gating step probably occurs in the circulation, where asparagine levels are low and are strongly affected by either L-asparaginase treatment or dietary restriction. Nonetheless, we do see effects on ratios of epithelial- to mesenchymal-like tumour cells at the primary and secondary site, which could also impact both tumour progression and response to therapy.

### Experimental Methods

### Cell Culture

The mouse mammary tumor cell line 4T1 (ATCC) and any derived clonal cell line were cultured in DMEM high glucose supplemented with 5% fetal bovine serum, 5% fetal calf serum, MEM non-essential amino acids (NEAA) and penicillin streptomycin (Thermo Fisher Scientific). The human breast tumor cell line MDAMB-231 (ATCC) was cultured in DMEM high glucose supplemented with 10% fetal bovine serum, NEAA and penicillin streptomycin (Thermo Fisher Scientific). The 4T1 and MDA-MB-231 cell lines were tested and authenticated by ATCC. The Platinum-A (Cell BioLabs) and 293FT (Thermo Fisher Scientific) packaging cell lines for virus production were cultured in DMEM high glucose supplemented with 10% fetal bovine serum and penicillin streptomycin. All cells lines have been routinely been tested for mycoplasma contamination.

### Virus Production

Retroviral vectors were packaged using the Platinum-A (Cell BioLabs) cell line and lentiviral vectors were packaged using the 293FT cell line (Thermo Fisher Scientific) as previously described in Wagenblast et al. 2015.

### Animal Studies

All mouse experiments were approved by the Cold Spring Harbor Animal Care and Use Committee. The maximal permitted tumour size of 20 mm in any direction was never exceeded. All mouse injections were carried out with 6-8-week-old female NOD-SCID-112rg^{-/-} (NSG) mice (JAX). Balb/c mice were not used in this study as the different clonal cell lines have variable GFP levels due to the lentiviral barcode vector. Tail vein injections were carried out using 5×10⁵ mouse mammary tumor cells, which were re-suspended in 100 ul of PBS and injected via tail vein. Orthotopic injections were performed using 1×10⁵ mouse mammary tumor cells or 5×10⁵ MDA-MB-231 cells. For this, cells were re-suspended in a 1:1 mix of PBS and growth factor reduced Matrigel (BD Biosciences). A volume of 20 ul was injected into the mammary gland #4 for mouse mammary tumor cells and a volume of 40 ul was injected for MDA-MB-231 cells. Primary tumor volume was measured using the formula V=½(L×W²), where L is length and W is width of the primary tumor. For L-asparaginase studies, mice were administered 200 ul of 60U of L asparaginase 5× per week through intraperitoneal injections. For L-asparagine adjusted diets, mice were given a control amino acid diet (0.6% asparagine), an asparagine deficient diet (0% asparagine) or an asparagine rich diet (4% asparagine). All diets were isonitrogenous and contained similar calorie densities Sample size was chosen to give sufficient power for calling significance with standard statistical tests. Mouse experiments were performed with 10 animals per condition to account for the variability that is observed in such in vivo experiments. Animals were assigned to treatment groups through random cage selection.

### Barcode Analysis

The barcodes of the 4T1-E and 4T1-T cells were amplified and sequenced as previously described in Wagenblast et al. 2015.

In Vivo shRNA Lung Screen and In Vitro Invasion Screen

shRNAs were predicted based on the Sherwood algorithm described in Knott et al. 2014. Pools of ˜50 shRNAs were packaged in Platinum-A cells. For each pool, 10 million 4T1-T cells were infected at a multiplicity of infection (MOI) of 0.3. The infected cells were selected with 500 ug/ml hygromycin for 5 days and each pool was injected into 5 mice each via the tail vein. A pre-injection pool was collected at the time of injection to validate equal representation of each shRNA. After 7 days, mice were sacrificed and perfused with PBS to remove blood and non-extravasated cells from the lungs. Lungs were harvested and genomic DNA was isolated using phenol chloroform extraction. Genomic DNA of the pre-injection pools was isolated using the QIAamp DNA Blood Mini Kit (Qiagen).

The in vitro invasion assays were carried out in parallel. Each pool was plated on two 6-well BioCoat Matrigel invasion plates (Corning). 6×10⁵ cells were plated on top of each well in cell culture media without serum. Cells were allowed to invade through the matrigel into media containing 5% fetal bovine serum and 5% fetal calf serum for 24 hours. Invaded cells were scraped off, washed with PBS and genomic DNA was isolated using the QIAamp DNA Blood Mini Kit.

The shRNAs were amplified using a two-step PCR protocol previously described in Knott et al. 2014 for next generation sequencing.

First PCR forward primer 1:
[SEQ ID NO: 1]
5-CAG AAT CGT TGC CTG CAC ATC TTG GAA AC-3
and
reverse primer 1:
[SEQ ID NO: 2]
5-CTG CTA AAG CGC ATG CTC CAG ACT GC-3.

Second PCR forward primer 2:
[SEQ ID NO: 3]
5- AAT GAT ACG GCG ACC ACC GAG ATC TAC ACT AGC
CTG CGC ACG TAG TGA AGC CAC AGA TGT A -3
and
reverse primer 2:
[SEQ ID NO: 4]
5- CAA GCA GAA GAC GGC ATA CGA GAT NNN NNN GTG
ACT GGA GTT CAG ACG TGT GCT CTT CCG ATC TCT GCT
AAA GCG CAT GCT CCA GAC TGC -3.
The reverse primer contained a barcode (NNNNNN)
that enabled multiplexing.

### Analysis of Screening Data

Each screen pool was analyzed as described in Knott et al. 2014.

### Gene Ontology Enrichment Analysis

Gene Ontology enrichment analysis was performed using the GOrilla web portal. The Refseq identifiers of genes identified as over-expressed in 4T1-T cells, as compared to 4T1-E cells, were used as foreground and the entire Refseq gene list was used as background.

### Expression Subtype and Relapse Analysis

All clinical data analysis was performed using the University of North Carolina “855 patient set”, which is available as published data at https://genome.unc.edu. All data was derived from an initial matrix that was arranged with patients on the horizontal axis and genes on the vertical axis. Initial normalization involved quantile normalization to ensure that the global expression profile of each patient was similar. Following this, each gene was z-score normalized across patients. For FIG. 1b, the average expression level of each gene was calculated for each patient subtype. For FIG. 1c, the average expression level of each gene was calculated for each gene for patients with and without relapse to each secondary site.

For FIG. 6a, the level of ASNS in each patient, stratified by subtype is plotted. For FIG. 6b, each boxplot represents the level of ASNS in each patient with and without relapse to each secondary site.

### Individual In Vitro Invasion Assay

The in vitro invasive capacity of cells was measured using 6-well BioCoat Matrigel invasion plates. For parental 4T1 cells, 1×10⁶ cells were plated on individual wells, for 4T1-T cells, 8×10⁵ cells were plated on individual wells and for MDA-MB-231 cells, 5×10⁵ cells were used per individual well. Cells were resuspended in media without serum and cells invaded into media with 5% fetal bovine serum and 5% fetal calf serum. For FIG. 3b and FIG. 9e, 4T1 and MDA-MB-231 cells were cultured in media containing 100× concentration of the specified amino acid (relative to the concentration in 1×NEAA) for 2 days or 3 days, respectively, before starting the invasion assay. After 24 h, non-invaded cells were removed and the invasion wells were washed in PBS, fixed in 2% glutalaldehyde for 2 min and stained with 0.5% crystal violet for 10 min. The wells were washed in distilled H₂O, air-dried and scanned using the Odyssey infrared scanner. The signal was quantified using ImageJ (NIH).

### Competition and Proliferation Assay

For the mCherry competition assay, shRNA-transduced mCherry-positive cells were admixed with untransduced cells. mCherry fluorescence was quantified on the LSR II flow cytometer (BD Biosciences). The proliferation assay was carried out using the CellTrace Violet Cell Proliferation Kit (Thermo Fisher Scientific). For FIG. 3b and FIG. 9e, 4T1 and MDA-MB-231 cells were cultured in media containing 100× concentration of the specified amino acid (relative to the concentration in 1×NEAA) for 2 days or 3 days, respectively, before starting the proliferation assay. Cells were stained with CellTrace violet and then trypsinized and re-suspended in media. After 24 hours, cells were collected in order to quantify violet fluorescence intensity using the SH800 flow cytometer (Sony).

### Isolation of Tumor and Lung Metastatic Sells

Tumor and lung tissue were harvested, minced and digested into single cells as previously reported in Wagenblast et al. 2015. Cells were either grown in 4T1 cell culture media containing 60 μM 6-thioguanine to deplete stromal cells or directly sorted based on mCherry expression using the FACSAria III cell sorter (BD Biosciences).

### RNAseq Library Preparation

RNAseq libraries from cultured 4T1-T cells were prepared in duplicates as previously described in Wagenblast et al. 2015. Each sample was sequenced on the Illumina HiSeq sequencer generating 76 nt single-end (SE) reads.

### RNAseq Analysis

Illumina sequencing reads were aligned to the mouse genome (mm10) using bowtie2 with default parameters (Langmead at al. 2012). Genes were assigned a count using HTseqcount (Anders et al. 2015). Differential expression analysis was performed using DESeq (Anders et al. 2010).

shRNA Knock-Down and cDNA Overexpression Studies

Mouse and human cell lines were transduced with shRNA expressing retroviral or lentiviral constructs, respectively. After infection, 4T1-T cells were selected with 500 ug/ml hygromycin for 5 days and MDA-MB-231 cells were selected with 2 ug/ml puromycin for 4 days. Cell lines infected with cDNA overexpressing retroviral constructs were selected with G418 for one week. The parental 4T1 cell line was selected with 600 ug/ml G418 and MDA-MB-231 cells were selected with 1500 ug/ml G418.

cDNA Overexpression Genes:
mouse ASNS: NM_012055.1
human ASNS: NM_001673.2
shRNA Knock-Down Sequences:

mouse shAsns-1 [SEQ ID NO: 5]:
TGCTGTTGACAGTGAGCGCCACTGCCAATAAGAAAGTATATAGTGAAGC
CACAGATGTATATACTTTCTTATTGGCAGTGTTGCCTACTGCCTCGGA
mouse shAsns-2 [SEQ ID NO: 6]:
TGCTGTTGACAGTGAGCGCCACTATGAAGTTTTGGATTTATAGTGAAGC
CACAGATGTATAAATCCAAAACTTCATAGTGTTGCCTACTGCCTCGGA
mouse shTgfb1-1 [SEQ ID NO: 7]:
TGCTGTTGACAGTGAGCGCCAGTATATATATGTTCTTCAATAGTGAAGC
CACAGATGTATTGAAGAACATATATATACTGTTGCCTACTGCCTCGGA
mouse shTgfb1-2 [SEQ ID NO: 8]:
TGCTGTTGACAGTGAGCGAAGTATATATATGTTCTTCAAATAGTGAAGC
CACAGATGTATTTGAAGAACATATATATACTGTGCCTACTGCCTCGGA
human shASNS-1 [SEQ ID NO: 9]:
TGCTGTTGACAGTGAGCGCCAGAAGCTAAAGGTCTTGTTATAGTGAAGC
CACAGATGTATAACAAGACCTITAGCTTCTGATGCCTACTGCCTCGGA
human shASNS-2 [SEQ ID NO: 10]:
TGCTGTTGACAGTGAGCGCAGCAATGACAGAAGATGGATATAGTGAAGC
CACAGATGTATATCCATCTTCTGTCATTGCTTTGCCTACTGCCTCGGA

qRT-PCR

Total RNA from cells was purified and DNAse treated using the RNeasy Mini Kit (Qiagen). For whole tissues, RNA was isolated using the TRIzol Plus RNA Purification Kit (Thermo Scientific). The tissue lysate was homogenized using a Dounce homogenizer and passed through a column homogenizer (Thermo Fisher Scientific) to reduce viscosity. RNA integrity (RNA Integrity score >9) was measured on the Agilent Bioanalyzer (RNA nano kit). cDNA was synthesized using SuperScript III Reverse Transcriptase (Sigma). Quantitative PCR analysis was performed on the Eppendorf Mastercycler ep realplex. All signals were quantified using the ΔCt method and were normalized to the levels of Gapdh. For mCherry-positive flow cytometer sorted tumor and lung metastatic cells, cDNA was produced directly from lysed cells using the TaqMan Gene Expression Cells-to-Ct Kit (Thermo Fisher Scientific). Quantitative PCR analysis was performed on the CFX96 (Bio-Rad) using TaqMan primer/probe sets and all signals were quantified as described above.

qRT-PCR Primers

mouse Asns (Exon 1-2)[SEQ ID NO: 11]:
5'-CCT CTG CTC CAC CTT CTC T-3' 5'-GAT CTT CAT
CGC ACT CAG ACA-3'
mouse Asns (Exon 6-7)[SEQ ID NO: 12]:
5'-CCA AGT TCA GTATCC TCT CCA G-3' 5'-CTT CAT
GAT GCT CGCTTC CA-3'
mouse Tgfb1 (Exon 1-2)[SEQ ID NO: 13]:
5'-CCG AAT GTC TGA CGT ATT GAA GA-3' 5'-GCG GAC
TAC TAT GCT AAA GAG G-3'
mouse Tgfb1 (Exon 3-4)[SEQ ID NO: 14]:
5'-GTT ATC TTT GCT GTC ACA AGA GC-3' 5-CCC ACT
GAT ACG CCT GAG-3'
mouse Gapdh (Exon 2-3)[SEQ ID NO: 15]:
5'-AAT GGT GAA GGT CGGTGT G-3' 5'-GTG GAGTCA
TACTGG AAC ATG TAG-3'
human ASNS (Exon 8-9)[SEQ ID NO: 16]:
5'-GAGTCA GAC CTT TGT TTA AAG CA-3' 5'-GGA GTG
CTT CAATGT AAC AAG AC-3'
human ASNS (Exon 12-13)[SEQ ID NO: 17]:
5'-CTG GAT GAA GTC ATATTT TCC TTG G-3' 5'-CAG
AGA AGATCA CCA CGCTAT C-3'
human GAPDH (Exon 2-3)[SEQ ID NO: 18]:
5'-ACA TCG CTC AGA CAC CAT G-3' 5'-TGT AGT TGA
GGT CAA TGA AGG G-3'

### TaqMan Probes:

### Mouse Asns: Mm00803785_m1

### Mouse E-cadherin (Cdh1): Mm01247357_m1

### Mouse Twist1: Mm00442036_m1

### Mouse Gapdh: Mm99999915_g1

qPCR for Circulating Tumor Cells (CTCs):

CTCs were quantified as previously described in Wagenblast et al. 2015. Genomic DNA was isolated from blood and quantified using a qPCR assay against mCherry, which is expressed from the retroviral shRNA delivery vectors.

```
mCherry probes and primers:
primer 1:
[SEQ ID NO: 19]
5'-GACTACTTGAAGCTGTCCTTCC-3'
primer 2:
[SEQ ID NO: 20]
5'-CGCAGCTTCACCTTGTAGAT-3'
HEX probe:
[SEQ ID NO: 21]
5'-/56-FAM/TTCAAGTGG/ZEN/GAGCGCGTGATGAA/
3IABkFQ//-3'
Housekeeping probes and primers:
primer 1:
    [SEQ ID NO: 22]
5'-GACTTGTAACGGGCAGGCAGATTGTG-3'
primer 2:
    [SEQ ID NO: 23]
5'-GAGGTGTGGGTCACCTCGACATC-3'
HEX probe:
[SEQ ID NO: 24]
5'-/5HEX/CCGTGTCGC/ZEN/TCTGAAGGGCAATAT/
3IABkFQ/-3'
```

### Quantification of Lung Metastasis

For each lung, five-micron sections were prepared and stained with a standard H&E protocol. Lung metastatic burden was determined by counting individual lung nodules on one section.

### E-Cadherin and Twist1 Analysis:

For immunohistochemistry, primary tumors and lungs were processed as previously described in Wagenblast et al. 2015. E-Cadherin (24E10) Rabbit mAb (3195, Cell Signaling) was utilized in a 1:400 dilution and Twist1 (Twist2C1a) Mouse mAb (ab50887, Abcam) was used in a 1:100 dilution. Ecadherin and Twist1 diaminobenzidine-stained (DAB) and hematoxylin stainings were quantified using ImageJ (NIH). For this, images were color deconvoluted according to Ruifrok et al. 2001 and the percent area of E-cadherin and Twist1 positive staining was measured.

### Free Amino Acid Quantification Using HPLC

Free amino acids were quantified in cultured cells and blood serum. For cultured cells, 4T1 and MDA-MB-231 cells were cultured in media containing 100× concentration of the specified amino acid (relative to the concentration in 1×NEAA) for 2 or 3 days, respectively. All cultured cells were homogenized using a Dounce homogenizer and the lysate was subsequently filtered. Each sample was quantified in triplicates using High Performance Liquid Chromatography (HPLC) and a flourometric detector. For each replicate, nanomoles of each amino acid was measured. The average of each triplicate was used to calculate the molar percent composition for each amino acid.

Proteomic Profiling Using Isobaric Tags for Relative and Absolute Protein Quantification (iTRAQ)

Cells were washed in ice cold PBS and harvested for iTRAQ quantitative proteomics as previously described in Ross et al. 2004. Three replicates were utilized for each cell line.

### Metabolite Profiling Using Liquid-Chromatography Tandem Mass Spectrometry (LC-MS/MS)

Metabolite extraction was performed as described previously (26). Organ tissue samples were placed in 2 ml lysing tubes prefilled with 1.4 mm ceramic beads for mammary glands or 2.8 mm ceramic beads for lungs and 1 ml of pre-chilled 80% methanol. Samples were homogenized with a Precellys24 homogenizer (Bertin Instruments) programmed with three 30 s cycles at 6500 Hz and 4 min pause times. At the end of each cycle, samples were snap-frozen in liquid nitrogen and placed on dry ice. Metabolite extraction of blood serum samples (50 ul) was performed using 200 ul of 80% methanol at −80 C. Following centrifugation for 10 min (13.2 kRPM, 4 C), supematants were evaporated to dryness and stored at −80 C until LC-MS/MS analysis.

Dried-down extracts were re-suspended in 25 ul HPLC-grade water, and 1 ul was analyzed using hydrophilic interaction chromatography (HILIC) coupled to tandem mass spectrometry analysis (LC-MS/MS). Analytical instrumentation consisted of a Nexera X2 (Shimadzu) liquid chromatography coupled to a QTRAP 6500 hybrid triple quadrupole/linear ion trap mass spectrometer (SCIEX) equipped with an electrospray ion source. Raw LC-SRM-MS data was acquired with Analyst 1.6.2 (SCIEX) and peak areas of LC-SRM-MS traces for each metabolite were integrated using the MultiQuant 1.1 software (SCIEX). Metabolite differences were analyzed by normalizing samples to total peak area and comparing replicates for each group using one-way ANOVA with multiple comparisons.

### Amino Acid Compositional Analysis

For FIG. 4a, transcriptional- and protein-level log-fold changes were quantile normalized to the same distribution. RNA-level changes were then subtracted from protein level changes. Amino acid representations in genes with the 10% highest and lowest subsequent values were then compared using a rank-sum test to identify amino acids whose abundance correlates with the protein-level changes that are not explained by transcriptional-changes. For FIG. 12b, amino acid representations were compared between genes that showed the greatest increase/decrease in RNA and protein expression using the same rank-sum test. For FIG. 12c & FIG. 12d the same analysis was performed, this time comparing the proteins of genes that had been detected as upregulated during EMT as compared to all other genes. In the case of FIG. 12c, the EMT-up genes were mouse orthologs of the EMT-up human genes.

For FIG. 12e, each organism harboring a minimum of 10 genes that were orthologs of the pro-EMT human genes described in FIG. 12d, were analyzed. For each organism, the asparagine percentage of each protein was calculated. Then the asparagine enrichment level for each organism was calculated by calculating the ratio of the median asparagine percentage of pro-EMT proteins vs. the remaining organism specific proteins. The statistical significance of enrichment was calculated as described for FIG. 12c and FIG. 12d.

### Ribosome Profiling Analysis

For analysis, quality trimming and linker removal was performed using cutadapt19. bowtie2 was used to remove reads that map to contaminating RNAs (e.g. rRNA and tRNA sequences)14. STAR was subsequently used to map reads of length 29-33 to the human transcriptome20. The offset was corrected for each read based on read length and 12 and 15 nucleotides downstream were marked as P and A sites. For each gene, we then calculated the number of events at all positions and aggregated the counts for each codon and subsequently amino acid.

### REFERENCES

Greaves, M. & Maley, C. C. Clonal evolution in cancer. Nature 481, 306-313 (2012).
Kang, Y. et al. A multigenic program mediating breast cancer metastasis to bone. Cancer cell 3, 537-549 (2003).
Lawson, D. A. et al. Single-cell analysis reveals a stem-cell program in human metastatic breast cancer cells. Nature 526, 131-135 (2015).
Lohr, J. G. et al. Widespread genetic heterogeneity in multiple myeloma: implications for targeted therapy. Cancer cell 25, 91-101 (2014).
Meacham, C. E. & Morrison, S. J. Tumour heterogeneity and cancer cell plasticity. Nature 501 (2013).
Mullighan, C. G. et al. Genomic analysis of the clonal origins of relapsed acute lymphoblastic leukemia. Science (New York, N.Y.) 322, 1377-1380 (2008).
Navin, N. et al. Tumour evolution inferred by single-cell sequencing. Nature 472 (2011).
Nguyen, A., Yoshida, M., Goodarzi, H. & Tavazoie, S. F. Highly variable cancer subpopulations that exhibit enhanced transcriptome variability and metastatic fitness. Nature communications 7, 11246 (2016).
Shackleton, M., Quintana, E., Fearon, E. R. & Morrison, S. J. Heterogeneity in cancer: cancer stem cells versus clonal evolution. Cell 138, 822-829 (2009).
Tirosh, I. et al. Dissecting the multicellular ecosystem of metastatic melanoma by single-cell RNA-seq. Science (New York, N.Y.) 352, 189-196 (2016).
Wang, Y. et al. Clonal evolution in breast cancer revealed by single nucleus genome sequencing. Nature 512, 155-160, doi:10.1038/nature13600 (2014).
Yu, M. et al. RNA sequencing of pancreatic circulating tumour cells implicates WNT signalling in metastasis. Nature 487, 510-513 (2012).
Wagenblast, E. et al. A model of breast cancer heterogeneity reveals vascular mimicry as a driver of metastasis. Nature 520, 358-362 (2015).
Vanharanta, S. & Massague, J. Origins of metastatic traits. Cancer cell 24, 410-421 (2013).
Miller, F. R., Miller, B. E. & Heppner, G. H. Characterization of metastatic heterogeneity among subpopulations of a single mouse mammary tumor: heterogeneity in phenotypic stability. Invasion & metastasis 3, 22-31 (1983).
Ashbumer, M. et al. Gene ontology: tool for the unification of biology. The Gene Ontology Consortium. Nature genetics 25, 25-29 (2000).
Harrell, J. C. et al. Genomic analysis identifies unique signatures predictive of brain, lung, and liver relapse. Breast cancer research and treatment 132, 523-535 (2012).
Knott, S. R. et al. A computational algorithm to predict shRNA potency. Molecular cell 56, 796-807 (2014).
Richards, N. G. & Schuster, S. M. Mechanistic issues in asparagine synthetase catalysis. Advances in enzymology and related areas of molecular biology 72, 145-198 (1998).
Richards, N. G. & Kilberg, M. S. Asparagine synthetase chemotherapy. Annual review of biochemistry 75, 629-654 (2006).
Tallal, L. et al. E. coli L-asparaginase in the treatment of leukemia and solid tumors in 131 children. Cancer 25, 306-320 (1970).
Ramakers-van Woerden, N. L. et al. TEL/AML1 gene fusion is related to in vitro drug sensitivity for L-asparaginase in childhood acute lymphoblastic leukemia. Blood 96, 1094-1099 (2000).
Stams, W. A. et al. Asparagine synthetase expression is linked with L asparaginase resistance in TEL-AML1-negative but not TEL-AML1-positive pediatric acute lymphoblastic leukemia. Blood 105, 4223-4225 (2005).
Loayza-Puch, F. et al. Tumour-specific proline vulnerability uncovered by differential ribosome codon reading. Nature 530, 490-494 (2016).
Taube, J. H. et al. Core epithelial-to-mesenchymal transition interactome gene-expression signature is associated with claudin-low and metaplastic breast cancer subtypes. Proceedings of the National Academy of Sciences of the United States of America 107, 15449-15454 (2010).
Langmead, B. & Salzberg, S. L. Fast gapped-read alignment with Bowtie 2. Nat. Methods 9, 357-359 (2012).
Anders, S., Pyl, P. T. & Huber, W. HTSeq—a Python framework to work with high-throughput sequencing data. Bioinformatics 31, 166-169 (2015).
Anders, S. & Huber, W. Differential expression analysis for sequence count data. Genome Biol. 11, R106 (2010).
Ruifrok, A. C. & Johnston, D. A. Quantification of histochemical staining by color deconvolution. Anal. Quant. Cytol. Histol. 23, 291-299 (2001).
Martin, M. Cutadapt removes adapter sequences from high-throughput sequencing reads. EMBnet.journal 17, 10 (2011).
Dobin, A. et al. STAR: ultrafast universal RNA-seq aligner. Bioinformatics 29, 15-21 (2013).
Ross, P. L. et al. Multiplexed protein quantitation in Saccharomyces cerevisiae using amine-reactive isobaric tagging reagents. Mol. Cell Proteomics 3, 1154-1169 (2004).

1. A dietary product comprising a plurality of amino acids, wherein the dietary product comprises all the essential amino acids and wherein the dietary product is substantially devoid of asparagine.

2. A dietary product in accordance with claim 1, wherein the dietary product comprises at least 12 amino acids.

3. A dietary product according to claim 1 to claim 2, wherein the dietary product is substantially devoid of at least one additional non-essential amino acid selected from the group consisting of: glutamine, glycine, serine, cysteine, tyrosine and arginine.

4. A dietary product according to any one of the preceding claims, wherein said dietary product further comprises one or more macronutrients and/or one or more micronutrients.

5. A dietary product according to any one of the preceding claims, wherein said dietary product further comprises methionine at a level of less than 25 mg/kg/day.

6. A dietary product in accordance with any preceding claim, wherein the product is formulated to provide at least the recommended daily intake of essential amino acids based on average daily total protein consumption.

7. A dietary product in accordance with any preceding claim, wherein the dietary product is in the form of a solid or fluid.

8. A process of preparing a dietary product in accordance with any preceding claims, wherein the components are dissolved or dispersed in water and spray dried.

9. A pharmaceutical composition comprising a dietary product in accordance with any one of claims 1 to 7 or a dietary product produced in accordance with claim 8 and a pharmaceutically acceptable carrier, excipient or diluent.

10. A pharmaceutical composition in accordance with claim 9, wherein the composition further comprises a therapeutic agent selected from: an inhibitor of cancer cell growth, an anti-metastatic agent, an immune checkpoint inhibitor, a radiotherapeutic agent and a chemotherapeutic agent.

11. A pharmaceutical composition in accordance with claim 10, wherein the therapeutic agent reduces asparagine levels in the blood.

12. The pharmaceutical composition of claim 11, wherein the therapeutic agent is an asparagine synthetase inhibitor or L-asparaginase.

13. A dietary product in accordance with any one of claims 1 to 7 or produced in accordance with claim 8 or a pharmaceutical composition in accordance with any one of claims 9-12 for use in therapy.

14. A medicament for use in delaying or inhibiting metastasis in a subject with cancer, wherein said medicament reduces asparagine levels in the blood of the subject with cancer.

15. The medicament for use in accordance with claim 14, wherein the medicament is selected from the group consisting of:
a. a dietary product in accordance with any one of claims 1 to 7;
b. a dietary product produced in accordance with claim 8;
c. a pharmaceutical composition in accordance with any one of claims 9-12;
d. an asparagine synthetase inhibitor; or
e. L-asparaginase.

16. The medicament for use in accordance with claim 14 or claim 15, wherein the cancer is selected from the group consisting of: breast cancer, colon cancer, squamous head and neck cancer, renal clear cell cancer and endometrial cancer.

17. The medicament for use in accordance with claim 15 or claim 16, wherein the dietary product or pharmaceutical composition is formulated for co-administration or sequential administration with L-asparaginase.

18. The medicament for use in accordance with any one of claims 15 to 17, wherein the dietary product is used in combination with a therapeutic agent selected from: an inhibitor of cancer cell growth, an anti-metastatic agent, an immune checkpoint inhibitor, a radiotherapeutic agent and a chemotherapeutic agent.

19. The medicament for use in accordance with any one of claims 14 to 18, wherein the subject has been determined to have an expression level of asparagine synthetase which is higher than a control or a predetermined level.

20. The medicament for use in accordance with any one of claims 14 to 19, wherein the subject has been determined to have a level of serum asparagine which is higher than a control or a predetermined level.

21. The medicament for use in accordance with any one of claims 14 to 20, wherein the subject has a solid tumour.

22. Use of a compound or composition in the manufacture of a medicament for delaying or inhibiting metastasis in a subject with cancer, wherein the compound or composition reduces extracellular asparagine levels in the blood of the subject.

23. Use in accordance with claim 22, wherein the compound or composition is:
a. dietary product in accordance with any one of claims 1 to 7;
b. a dietary product produced in accordance with claim 8;
c. a pharmaceutical composition in accordance with any one of claims 9-12;
d. an asparagine synthetase inhibitor; or
e. L-asparaginase.

24. Use in accordance with claim 22 or claim 23, wherein the cancer is selected from the group consisting of: breast cancer, colon cancer, squamous head and neck cancer, renal clear cell cancer and endometrial cancer.

25. Use in accordance with claim 23 or claim 24, wherein the dietary product or pharmaceutical composition is formulated for co-administration or sequential administration with L-asparaginase.

26. Use in accordance with any one of claims 23 to 25, wherein the dietary product is used in combination with a therapeutic agent selected from: an inhibitor of cancer cell growth, an anti-metastatic agent, an immune checkpoint inhibitor, a radiotherapeutic agent and a chemotherapeutic agent.

27. Use in accordance with any one of claims 22 to 26, wherein the subject has been determined to have an expression level of asparagine synthetase which is higher than a control or a predetermined level.

28. Use in accordance with any one of claims 22 to 27, wherein the subject has been determined to have a level of serum asparagine which is higher than a control or a predetermined level.

29. Use in accordance with any one of claims 22 to 28, wherein the subject has a solid tumour.

30. A method of treating cancer in a subject, comprising administering a therapeutically effective amount of a medicament to said subject, wherein said medicament reduces asparagine levels in the blood of the subject with cancer.

31. The method according to claim 30, wherein the medicament comprises:
a. a dietary product in accordance with any one of claims 1 to 7;
b. a dietary product produced in accordance with claim 8;
c. a pharmaceutical composition in accordance with any one of claims 9-12;
d. an asparagine synthetase inhibitor; or
e. L-asparaginase.

32. The method in accordance with claim 30, wherein the cancer is selected from the group consisting of: breast cancer, colon cancer, squamous head and neck cancer, renal clear cell cancer and endometrial cancer.

33. The method in accordance with claim 31, wherein a therapeutically effective amount of the dietary product or the pharmaceutical composition is co-administered or sequentially administrated with a therapeutically effective amount of L-asparaginase.

34. The method in accordance with claim 31, wherein a therapeutically effective amount of the dietary product is administered in combination with a therapeutic agent selected from: an inhibitor of cancer cell growth, an anti-metastatic agent, an immune checkpoint inhibitor, a radiotherapeutic agent and a chemotherapeutic agent.

35. The method in accordance with claim 30, wherein the subject has been determined to have an expression level of asparagine synthetase which is higher than a control or a predetermined level.

36. The method in accordance with claim 30, wherein the subject has been determined to have a level of serum asparagine which is higher than a control or a predetermined level.

37. The method in accordance with claim 30, wherein the subject has a solid tumour.

38. The method in accordance with claim 31, wherein the dietary product is the sole source of nutrition for the subject.

39. The method in accordance with claim 30, wherein the treatment is administered over a period of at least 24 hours or until a therapeutic endpoint is observed.

40. The method in accordance with claim 30, wherein the medicament is administered between 1 and 6 times a day.

41. The method in accordance with claim 31, wherein at least the recommended daily amount of essential amino acids are met by the administration regimen of the dietary product each day.

42. Use of serum asparagine levels as a biomarker to identify a patient or patient population having a tumour which is at increased risk of metastasis.

43. Use of asparagine synthetase expression as a biomarker to identify a patient or patient population having a tumour which is at increased risk of metastasis.

44. A method of identifying a subject with cancer having an increased likelihood of metastasis comprising:
a) determining the level of asparagine in a biological sample isolated from the subject;
b) comparing the level of asparagine in the biological sample to a control sample or to a predetermined reference level of asparagine,
wherein an increased level of asparagine in the biological sample compared to the control sample or compared to the predetermined reference level is indicative of increased likelihood of metastasis.

45. The method of claim 44, further comprising administering a therapeutically effective amount of a medicament to said subject, wherein said medicament reduces asparagine levels in the blood of the subject with cancer.

46. The method according to claim 45, wherein the medicament is:
a. a dietary product in accordance with any one of claims 1 to 7;
b. a dietary product produced in accordance with claim 8;
c. a pharmaceutical composition in accordance with any one of claims 9-12;
d. an asparagine synthetase inhibitor; or
e. L-asparaginase.

47. A method of identifying a subject having an increased likelihood of responsiveness or sensitivity to a cancer treatment when fed a diet substantially devoid of asparagine or administered with L-asparaginase comprising:
a) determining the level of asparagine in a biological sample isolated from the subject;
b) comparing the level of asparagine in the biological sample to a control sample or to a predetermined reference level of asparagine,
wherein an increased level of asparagine in the biological sample compared to the control sample or compared to the predetermined reference level is indicative of responsiveness or sensitivity to said cancer treatment when combined with a diet substantially devoid of asparagine or administered with L-asparaginase.

48. The method of claim 47, further comprising administering a therapeutically effective amount of the dietary product in accordance with any one of claims 1 to 7 or the dietary product produced in accordance with claim 8 or the pharmaceutical composition in accordance with any one of claims 9-12 or L-asparaginase in combination with a chemotherapeutic agent the subject is identified as having an increased likelihood of responsiveness or sensitivity to a cancer treatment when fed a diet substantially devoid of asparagine or administered with L-asparaginase.

49. The method according to claim 44 or claim 47, where in the biological sample is blood serum.

50. A method of determining the likelihood of metastatic relapse in a subject having a cancer comprising:
a) determining the level of asparagine synthetase in a biological sample isolated from the subject;
b) comparing the level of asparagine synthetase in the biological sample to a control sample or to a predetermined reference level of asparagine,
wherein an increased level of asparagine synthetase in the biological sample compared to the control sample or compared to the predetermined reference level is indicative of an increased likelihood of metastatic relapse.

51. A method of reversing epithelial to mesenchymal transition in a subject or prevent epithelial to mesenchymal transition in a subject with cancer comprising administering a therapeutically effective amount of the dietary product in accordance with any one of claims 1 to 7 or the dietary product produced in accordance with claim 8 or the pharmaceutical composition in accordance with any one of claims 9-12 or L-asparaginase to a subject in need thereof.
